SEQUENCE LISTING (26 sequences)

SEQ ID NO: 1 - DNA, 145 bp, synthetic construct
  source 1..145: mol_type = other DNA
tatgaagtgttgtggttccttaaccacatttctttttttttttttccaggctattcaagatctctggcagtggaggaagtctctttaagaaaatagtttaaacaatttgttaaaaaattttccgtcttatttcatttctgtaaca

SEQ ID NO: 2 - DNA, 149 bp, synthetic construct
  source 1..149: mol_type = other DNA
  misc_difference 67..70replace
tatgaagtgttgtggttccttaaccacatttctttttttttttttccaggctattcaagatctctgtctggcagtggaggaagtctctttaagaaaatagtttaaacaatttgttaaaaaattttccgtcttatttcatttctgtaaca

SEQ ID NO: 3 - DNA, 149 bp, synthetic construct
  source 1..149: mol_type = other DNA
  misc_difference 67..70replace
tatgaagtgttgtggttccttaaccacatttctttttttttttttccaggctattcaagatctctgcatggcagtggaggaagtctctttaagaaaatagtttaaacaatttgttaaaaaattttccgtcttatttcatttctgtaaca

SEQ ID NO: 4 - DNA, 149 bp, synthetic construct
  source 1..149: mol_type = other DNA
  misc_difference 67..70replace
tatgaagtgttgtggttccttaaccacatttctttttttttttttccaggctattcaagatctctgcctggcagtggaggaagtctctttaagaaaatagtttaaacaatttgttaaaaaattttccgtcttatttcatttctgtaaca

SEQ ID NO: 5 - DNA, 24 bp, synthetic construct
  source 1..24: mol_type = other DNA
tatgaagtgttgtggttccttaac

SEQ ID NO: 6 - DNA, 25 bp, synthetic construct
  source 1..25: mol_type = other DNA
tgttacagaaatgaaataagacgga

SEQ ID NO: 7 - DNA, 21 bp, synthetic construct
  source 1..21: mol_type = other DNA
gactgaccaagaggctattca

SEQ ID NO: 8 - DNA, 25 bp, synthetic construct
  source 1..25: mol_type = other DNA
tgttacagaaatgaaataagacgga

SEQ ID NO: 9 - DNA, 333 bp, synthetic construct
  source 1..333: mol_type = other DNA
ctttatctagagttaactctctggtggtagaatgaaaaatagatgttgaactatgcaaagagacatttaatttattgatgtctatgaagtgttgtggttccttaaccacatttctttttttttttttccaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 10 - DNA, 337 bp, synthetic construct
  source 1..337: mol_type = other DNA
  misc_difference 149..152replace
ctttatctagagttaactctctggtggtagaatgaaaaatagatgttgaactatgcaaagagacatttaatttattgatgtctatgaagtgttgtggttccttaaccacatttctttttttttttttccaggctattcaagatctctgtctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 11 - DNA, 337 bp, synthetic construct
  source 1..337: mol_type = other DNA
  misc_difference 149..152replace
ctttatctagagttaactctctggtggtagaatgaaaaatagatgttgaactatgcaaagagacatttaatttattgatgtctatgaagtgttgtggttccttaaccacatttctttttttttttttccaggctattcaagatctctgcatggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 12 - DNA, 337 bp, synthetic construct
  source 1..337: mol_type = other DNA
  misc_difference 149..152replace
ctttatctagagttaactctctggtggtagaatgaaaaatagatgttgaactatgcaaagagacatttaatttattgatgtctatgaagtgttgtggttccttaaccacatttctttttttttttttccaggctattcaagatctctgcctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 13 - DNA, 379 bp, synthetic construct
  source 1..379: mol_type = other DNA
ggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 14 - DNA, 383 bp, synthetic construct
  source 1..383: mol_type = other DNA
  misc_difference 194..197replace
ggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgtctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 15 - DNA, 383 bp, synthetic construct
  source 1..383: mol_type = other DNA
  misc_difference 194..197replace
ggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgcatggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 16 - DNA, 383 bp, synthetic construct
  source 1..383: mol_type = other DNA
  misc_difference 194..197replace
ggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctgcctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgc

SEQ ID NO: 17 - DNA, 13 bp, synthetic construct
  source 1..13: mol_type = other DNA
  modified_base order(5,8..10): mod_base = OTHER; note = locked nucleotides
actgccagagatc

SEQ ID NO: 18 - DNA, 11 bp, synthetic construct
  source 1..11: mol_type = other DNA
  modified_base order(3,6..8,10): mod_base = OTHER; note = locked nucleotides
tgccagacaga

SEQ ID NO: 19 - DNA, 11 bp, synthetic construct
  source 1..11: mol_type = other DNA
  modified_base order(3,5..7,9): mod_base = OTHER; note = locked nucleotides
tgccatgcaga

SEQ ID NO: 20 - DNA, 11 bp, synthetic construct
  source 1..11: mol_type = other DNA
  modified_base order(5..7,9..10): mod_base = OTHER; note = locked nucleotides
tgccatgcaga

SEQ ID NO: 21 - DNA, 11 bp, synthetic construct
  source 1..11: mol_type = other DNA
  modified_base 6..8: mod_base = OTHER; note = locked nucleotides
tgccaggcaga

SEQ ID NO: 22 - DNA, 10 bp, synthetic construct
  source 1..10: mol_type = other DNA
tgccagagat

SEQ ID NO: 23 - DNA, 12 bp, synthetic construct
  source 1..12: mol_type = other DNA
  modified_base order(3,7..9,11): mod_base = OTHER; note = locked nucleotides
ctgccagacaga

SEQ ID NO: 24 - DNA, 26 bp, synthetic construct
  source 1..26: mol_type = other DNA
  modified_base order(4,9,18..20): mod_base = OTHER; note = locked nucleotides
gctgcacgtcatcgatcatctcatgc

SEQ ID NO: 25 - DNA, 26 bp, synthetic construct
  source 1..26: mol_type = other DNA
  modified_base order(4,9,18..20): mod_base = OTHER; note = locked nucleotides
gctgcacgtcatcgatcatctcatgc

SEQ ID NO: 26 - DNA, 26 bp, synthetic construct
  source 1..26: mol_type = other DNA
gctgcacgtcatcgatcatctcatgc

(19) United States
(12) Patent Application Publication
(10) Pub. No.: US 2024/0336978 A1
(43) Pub. Date: Oct. 10, 2024

(54) COMPOSITIONS AND METHODS FOR FAST MULTIPLEXED SCREENING AND MONITORING OF NPM1 MUTATIONS

(71) Applicant: The University of Hong Kong, Hong Kong (CN)

(72) Inventors: Gill Harinder Harry Singh, Hong Kong (CN); Rita Lok Hay Yim, Hong Kong (CN); Paul Lee, Hong Kong (CN)

(21) Appl. No.: 18/681,271

(22) PCT Filed: Aug. 5, 2022
(86) PCT No.: PCT/CN2022/110519

§ 371 Date: Feb. 5, 2024

Related U.S. Application Data
(60) Provisional application No. 63/229,657, filed on Aug. 5, 2021.

(51) Int. Cl. C12Q 1/6886; C12Q 1/6816

(52) U.S. Cl. CPC C12Q 1/6886; C12Q 1/6816; C12Q 2600/156

(57) ABSTRACT

Compositions and methods for the reliable molecular monitoring of NPM1 mutations are disclosed. The methods enable pre-emptive therapy essential for understanding AML disease status, informing therapeutic options, monitoring therapeutic efficacy, and improving patient outcome. The methods simultaneously screen and quantify samples having the major NPM1 mutant subtypes. In some forms, methods for detecting and measuring a presence, absence, and/or level of mutation of the NPM1 gene in a sample include hybridizing labelled sequence probes to the NPM1 gene in a sample to form labelled probe-nucleic acid conjugates, amplifying the probe-nucleic acid conjugates, and detecting the labelled probe-nucleic acid conjugates by measuring the level of one or more labels attached to the probes. The methods optionally record the presence, absence and/or level of NPM1 mutations in the sample.

### CROSS-REFERENCE TO RELATED APPLICATION

The present application claims priority to U.S. Application No. 63/229,657 filed Aug. 5, 2021, the disclosure of which is incorporated herein by reference in its entirety.

### FIELD OF THE INVENTION

The invention is generally directed to molecular systems for sequencing and characterizing nucleic acids, and in particular, simultaneous screening and absolute quantification of mutations in proto-oncogenes associated with leukemia.

### BACKGROUND OF THE INVENTION

Acute Myeloid Leukemia (AML) is a blood cancer that is initiated from immature white blood cells (granulocytes or monocytes) in the bone marrow and is amongst the most common form of leukemia in adults. AML accounts for about 1% of all cancers is, with around 20,000 new cases per year in the USA. Mutations in the nucleophosmin 1 (NPM1) gene occur in one third of all patients with AML and in approximately 50 percent of patients with cytogenetically normal AML (Papaemmanuil, et al., N Engl J Med. 2016; 374 (23): 2209-21; Ley, et al., N Engl J Med. 2013; 368 (22): 2059-74; and Patel, et al. N Engl J Med. 2012; 366 (12): 1079-89). More than 50 different mutations on exon 12 of the NPM1 gene have been described, however more than 95% of cases involve one of three frameshift mutations termed types A, B and D (Falini, et al., Blood. 2007; 109 (3): 874-85; Rau and Brown, Hematol Oncol. 2009; 27 (4): 171-81). FMS-like tyrosine kinase 3 internal tandem duplication (FLT3-ITD) or DNMT3A mutation occur in approximately 66% of patients with NPM1-mutated AML and are associated with a poor prognosis and are common indications for allogeneic hematopoietic stem cell transplantation (HSCT) in eligible patients. Current treatment regimens include allogeneic hematopoietic stem cell transplantation (HSCT) in eligible patients, as well as chemotherapy (Schlenk, et al., N Engl J Med. 2008; 358 (18): 1909-18; Ley, et al, N Engl J Med. 2010; 363 (25): 2424-33; Metzeler, et al., Leukemia. 2012; 26 (5): 1106-7; Gale, et al., J Clin Oncol. 2015; 33 (18): 2072-83; Peterlin, et al., Haematologica. 2015; 100 (5): e196-9). However, persistence of NPM1 mutant transcripts following induction or consolidation chemotherapy is associated with high relapse rates (Ivey, et al., N Engl J Med. 2016; 374 (5): 422-33; Balsat, et al., J Clin Oncol. 2017; 35 (2): 185-93). In high-risk NPM1-mutated AML patients undergoing allogeneic HSCT, measurable residual disease (MRD) positivity prior to HSCT was associated with high risk of post-HSCT relapse (Dillon, et al., Blood. 2020; 135 (9): 680-8). Therefore, novel strategies for molecular monitoring and pre-emptive therapy are necessary to improve patient outcome.

Current strategies of molecular monitoring are limited by their sensitivities, dependence on RNA quality, turnaround-time (TAT) and cost. In addition, there is lack of a platform that allows simultaneous screening of the major mutant subtype and quantification. Conventional methods of detecting and classifying (or screening) requires Sanger sequencing which is highly labor- and time-intensive. Furthermore, because this system requires manual examination of the raw sequence data, results are often misinterpreted. At diagnosis, next generation sequencing (NGS) is often used for quantifying mutations of a large number of additional genes. This method is expensive and not a validated means for subsequent minimal residual disease (MRD) monitoring.

Commercial options based on Real Time polymerase chain reaction (PCR) are not cost effective and require large amounts of potentially unstable ribonucleic acid (RNA) from patients as input material are available. Further, these systems suffer from limited sensitivity due to the stringent RNA quality requirements, and are not specific enough to effectively provide mutation subtyping at diagnosis (see U.S. Pat. No. 10,526,650; European Patent No. 2518164B1; International Publication No. WO 2017201276; Bacher, et al., British Journal of Haematology 167, 710-714, 2014 (doi: 10.1111/bjh.13038); Mencia-Trinchant, et al., The Journal of Mol Diagnostics 19, 537-548, 2017, doi: 10.1016/j.jmoldx.2017.03.005).

There is a need to develop more sensitive, specific and cost-effective means of detecting NPM1 mutations in AML. There is also a need for high-throughput assays for quantifying NPM1 mutations with high sensitivities and specificities that do require RNA as an input material.

Therefore, it is an object of the invention to provide compositions and methods of use thereof for sensitive, automated detection of NPM1 mutations in AML with enhanced accuracy.

It is another object to provide compositions and methods for simultaneous screening and absolute quantification of the four major NPM1 mutation subtypes.

It is a further object of the invention to provide compositions and methods for rapid, high-throughput screening for NPM1 mutations based on DNA as an input material.

It is another object to provide compositions and methods for direct comparison with next generation sequencing results that saves additional time and cost to determine baseline value for minimal residual disease (MRD) monitoring.

### SUMMARY OF THE INVENTION

A multiplexed probe-based PCR system for detecting mutations in the NPM1 gene has been developed. Compositions and methods for simultaneous screening and absolute quantification of the three major NPM1 mutation subtypes (A, B and D) versus wild type NPM1 are described. The systems employ a modified oligonucleotide probe that incorporates Locked Nucleic Acid (LNA) to provide enhancing accuracy of screening.

Methods for detecting and/or measuring a presence, absence and/or level of mutation of the NPM1 gene in a sample are provided. The methods include the steps of (a) hybridizing labelled sequence probes to the NPM1 gene in a sample to form probe-nucleic acid conjugates; (b) amplifying the probe-nucleic acid conjugates; and (c) detecting the amplified probe-nucleic acid conjugates. In some forms, the methods include one or more additional steps of (d) recording the presence, absence and/or level of NPM1 mutations in the sample.

In some forms, the process of hybridizing in step (a) includes (i) combining in a reaction mixture under primer extension conditions a set of oligonucleotide primers with the sample; and (ii) adding to the reaction mixture one or more labelled sequence probes. The primers include sequences complementary to the NPM1 gene and are configured to produce an extension product comprising all or part of the NPM1 gene.

In preferred forms, each probe includes one or more Locked Nucleic Acid (LNA). The probes have a nucleic acid sequence complementary to the NPM1 gene, or to a mutant of the NPM1 gene; and a detectable label, preferably a reporter dye, and a quencher dye pair. Typically, the sequence complementary to the NPM1 gene anneals to the extension product at a predetermined location. In some forms the reagents are carried out within a single reaction mixture. In some forms the methods are carried out within a single reaction vessel.

In preferred forms, the mutation of the NPM1 gene is one or more frameshift mutations in the NPM1 gene. Exemplary frameshift mutations include type A NPM1 mutation, type B NPM1 mutation, and type D NPM1 mutation of NPM1. In some forms, the extension product obtained in step (a)(i) includes all or part of intron 12 of the human NPM1 gene. In some forms the set of oligonucleotide primers includes two oligonucleotide primers having the nucleic acid sequences of SEQ ID NO:5 and SEQ ID NO:6, respectively. In other forms the set of oligonucleotide primers includes two oligonucleotide primers having the nucleic acid sequences of SEQ ID NO:7 and SEQ ID NO: 8, respectively.

In preferred forms amplifying the probe-nucleic acid conjugates in (b) includes performing a polymerase chain reaction (PCR) in the reaction mixture to form an amplicon using Real Time PCR, Digital PCR, or Droplet-Digital PCR. In some forms the sample includes a nucleic acid plasmid comprising all or part of the NPM1 gene, genomic DNA, cDNA. In some forms the method includes the use of one or more controls. For example, in some forms determining the presence, absence and/or level of NPM1 mutations in the sample in (d) includes comparing the results from a sample with those obtained from the one or more controls. In preferred forms, one control includes a nucleic acid sequence corresponding to that of wild-type NPM1, type A NPM1 mutation, type B NPM1 mutation, or type D NPM1 mutation.

In some forms, the labelled sequence probes include four probes, including a first probe having a nucleic acid sequence complementary to that of wild-type NPM1, a second probe having a nucleic acid sequence complementary to that of NPM1 type A mutation, a third probe having a nucleic acid sequence complementary to that of NPM1 type B mutation, and a fourth probe having a nucleic acid sequence complementary to that of NPM1 type D mutation. In some forms one reporter dye is associated with one mutation. Exemplary reporter dyes include FAM, HEX, TAMRA, ROX, ATTO550, TEX615, Cy5 and TYE665. Preferably, one or more probes includes a nucleic acid sequence of one or more of SEQ ID NO: 17, 18, 19, 20, 21, 22, and 23.

Typically, the detecting in step (c) requires the presence of 100 copies, or less than 100 copies of a target NPM1 nucleic acid. In some forms, the detecting in step (c) requires 10 copies, or less than 10 copies of a target NPM1 nucleic acid. In particular forms, the detecting in step (c) requires 5 copies, or less than 5 copies of a target NPM1 nucleic acid.

In particular forms, subject is diagnosed as having Acute Myeloid Leukemia (AML) or is identified as being at risk of AML. In some forms, the methods further include one or more steps of administering to the subject a therapeutic agent or surgery. Exemplary therapeutic agents include chemotherapeutic agents and antibiotic agents. Exemplary surgical procedures include hematopoietic stem cell therapy (HSCT). Therefore, in some forms the subject is undergoing, or is eligible for chemotherapy. In other forms the subject is undergoing or is eligible for hematopoietic stem cell therapy (HSCT). In some forms the methods are used to detect or quantify Measurable Residual Disease (MRD) in the subject.

Kits for detecting and measuring a presence, absence and/or level of mutations of the NPM1 gene in a sample are also provided. Exemplary kits include one or more reagents including (i) oligonucleotide primers; and (ii) one or more labelled sequence probes. The kits optionally further include (iii) instructions for a method of detecting and measuring a presence, absence and/or level of mutation of the NPM1 gene in a sample.

Typically, the primers include nucleic acid sequences complementary to the NPM1 gene and are configured to produce an extension product including all or part of the NPM1 gene.

Typically, the probes include a nucleic acid sequence complementary to wildtype NPM1 or a mutant of the NPM1, which anneals to the extension product at a predetermined location; and a reporter dye and a quencher. Preferably the probes include Locked nucleic Acid (LNA).

### BRIEF DESCRIPTION OF THE DRAWINGS

FIG. 1A is a schematic illustration of the NPM1 gene wild type, and mutants. FIG. 1B is a schematic of a multiplexed probe-based PCR system for detecting NPM1 mutations in AML using a four or more-channel configuration FIG. 1C is a schematic of a two-channel configuration probe-based PCR system for detecting NPM1 mutations in AML. The NPM1 mutation detection assay with multiplexed LNA probes targeting type A NPM1 mutation (designated as Mutant A or Mut A), type B NPM1 mutation (designated as Mutant B or Mut B) and type D NPM1 mutation (designated as Mutant D or Mut D) is depicted using either a Genomic NPM1 DNA, or NPM1 transcript prepared from RNA. Two sets of PCR primers (half arrow) are depicted as being used for the DNA and RNA samples, respectively. Four LNA probes targeting the corresponding NPM1 mutants are designed with different reporter dye labels at 5′ end and quenchers at 3′ end. FIG. 1D shows the structure of locked Nucleic acid.

FIGS. 2A-2F are graphs showing the amplification plot of LNA probes using 4-plex Real Time (RT) PCR. Graphs show ΔRn over Cycle number for tracers corresponding to probes specific for Mutant A, Mutant B, Mutant D and Wild type, respectively for each of Mutant A (FAM) channel (FIG. 2A); Mutant B (TAMRA) channel (FIG. 2B); Wild type (HEX) channel (FIGS. 2C-2D); and Mutant D (ROX) channel (FIGS. 2E-2F), respectively.

FIGS. 3A-3E are graphs showing the amplification of probes using 4-plex Real Time (RT) PCR. Graphs show ΔRn over Cycle number for tracers corresponding to probes specific for Mutant A, Mutant B, Mutant D and Wild type, respectively for each of Mutant A (FAM) channel (FIG. 3A); Mutant B (FAM) channel (FIG. 3B); Wild type (HEX) channel (FIG. 3C); and Mutant D (FAM) channel (FIG. 3D), and Mutant D (ROX) channel (FIG. 3E) respectively.

FIGS. 4A-4C are scatter plots showing the 2D visualization of positive events (Fluorescence intensity, RFU) of wild-type NPM1 and three types of mutants in collective plasmid controls and patient samples, for each of Mutant A (Mut A) (FIG. 4A); Mutant B (Mut B) (FIG. 4B); and Mutant D (Mut D) (FIG. 4C), respectively. The positions of positive events for wild type and for each of the mutants within each graph are indicated.

FIGS. 5A-5C are panels of scatter plot graphs showing data obtained in individual digital PCR wells from an assay carried out on samples from each of two patients (A and B), using Mutant A-FAM, Mutant B-TAMARA, Mutant D-ROX and WildType-HEX probes, as well as a reference detector.

FIG. 6 is a scatter plot graph showing data obtained in a digital QIACUITY® PCR from an assay carried out using Mutant A (Mut A); Mutant B2 (Mut B2); Mutant B3 (Mut B3); Mutant D (Mut D); and WildType (WT+) FAM probes.

FIG. 7 is a panel of scatter plot graphs showing Amplitude over Event number obtained in each FAM or HEX channel when loaded with Mutant B (Mut B) or Mutant D (Mut D) plasmids and probed with Mutant B2 (Mut B2); Mutant B3 (Mut B3), Mutant D (Mut D); and Wild-Type (WT+) FAM or HEX probes.

FIGS. 8A-8D are 2D scatter plot graphs showing Chanel 1 Amplitude over Channel 2 Amplitude for each of FAM and HEX channels when loaded with Mutant B2 (Mut B2) Radial Multiplex (FIG. 8A); Mutant B3 (Mut B3) Radial Multiplex (FIG. 8B); or Mutant D (Mut D) Radial Multiplex (FIG. 8C); and WildType (WT) Radial Multiplex (FIG. 8D), respectively.

FIGS. 9A-9C are graphs showing the Standard curve of the log linearity of calculated abundance (Ct values) with increasing dilution (Quantity) for each of WildType (FIG. 9A), Mutant B3 (FIG. 9B), and Mutant A (FIG. 9C) NPM1 respectively, as determined by Real Time PCR.

FIG. 10 is a bar graph showing Absolute counts for Mutant A (Mut A) and NPM1 Wild Type (NPM1 Wt) in each of 4 test wells (A3-D3), respectively. Data are listed in tabular form in Table 2.

FIGS. 11A-11C are bar graphs showing Concentration (copies/μl) for each of Mutant B (Mut B) (FIG. 11A), Mutant D (Mut D) (FIG. 11B), and NPM1 Wild Type (NPM1 Wt) (FIG. 11C), in each of 4 partitions (10%, 1%, 0.5% and 0.1%), respectively. Data are listed in tabular form in Table 3.

FIGS. 12A-12C are graphs showing Measured mutation Burden (%) over Expected mutation Burden (%) for each of Mutant B2 (Mut B), (FIG. 12A), Mut B3 (FIG. 12B) and Mutant D (Mut D) (FIG. 12C), respectively, using plasmid controls mimicking patient DNA on ddPCR.

FIGS. 13A-C show a correlation study of mutant A abundance at DNA level by dPCR/ddPCR with RNA abundance using Real Time PCR.

FIG. 14A shows serial timepoint of Patient B displaying molecular monitoring and detection applications in both DNA and RNA. FIG. 14B shows serial timepoint of Patient B (DNA) running on QX200.

FIG. 15 shows mutation burden of NPM1 in patients under stable condition or remission compared with relapsed or refractory to treatment.

FIGS. 16A-16C are graphs showing DNA visualization traces of individual dPCR wells for Mutant A signal (FIG. 16A), Mutant B Signal (FIG. 16B), and Mutant D Signal (FIG. 16C). FIG. 16D is a graph showing a RNA visualization trace of the Mutant B Signal for a non-ABD patient at relapse.

FIGS. 17A and 17B are graphs showing results from the LOD testing of all Mutant probes using the probe for Mutant B on QX200 and as low as 1% mutant copy can be detected in 200 copy/uL of WT control at DNA level.

### DETAILED DESCRIPTION OF THE INVENTION

### I. Definitions

The terms “NPM1”, or “NPM1 gene” refer to the human nucleophosmin 1 gene. An exemplary nucleic acid sequence for the human nucleophosmin 1 gene is set forth in GenBank accession no. NC_000005.10 (region 171387116 . . . 171410900). A fragment of exon 12 of the NPM1 gene is set forth in SEQ ID NO:1.

The terms “NPM1 Mutant”, or “NPM1 mutation” refer to one of the three most common frameshift mutations in the human NPM1 gene that are associated with Acute Myeloid Leukemia (AML). These include mutations designated “A”, “B”, and “D”. Exemplary nucleic acid sequences for each of mutants A, B, and D include SEQ ID NO: 2, 3 and 4, respectively.

The term “Locked Nucleic Acid”, or “LNA” refers to a modified RNA monomer, having a methylene bridge bond linking the 2′ oxygen to the 4′ carbon of the RNA pentose ring. The bridge bond fixes the pentose ring in the 3′-endo conformation.

The term “nucleic acid sample” refers to a composition, such as a solution, that contains or is suspected of containing nucleic acid molecules.

The term “DNA sample” refers to a composition, such as a solution, that contains or is suspected of containing Deoxyribonucleic Acid molecules.

The term “nucleotide” refers to a molecule that contains a base moiety, a sugar moiety, and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The term “oligonucleotide” or a “polynucleotide” are synthetic or isolated nucleic acid polymers including a plurality of nucleotide subunits.

The terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with cancer are mitigated or eliminated, including, but are not limited to, reducing and/or inhibiting rate of tumor cell proliferation/growth, increasing the quality of life of those suffering from the disease, decreasing the dose of other medications required to treat the disease, delaying the progression of the disease, and/or prolonging survival of individuals.

The terms “individual,” “host,” “subject,” and “patient” are used interchangeably, and refer to a mammal, including, but not limited to, murines, simians, humans, mammalian farm animals, mammalian sport animals, and mammalian pets.

“Quenchers are used herein are substances capable of absorbing energy from a fluorophore (such as a fluorescent dye) and re-emitting much of that energy as either heat (in the case of dark quenchers) or visible light (in the case of fluorescent quenchers). Dabcyl is an example of a dark quencher, and TAMRA is an example of a fluorescent quencher.

### II. Compositions

It has been demonstrated that multiplexed probe-based PCR systems can simultaneously detect and effectively quantify NPM1 mutations in AML. The systems employ a modified probe that incorporates Locked Nucleic Acid (LNA) to provide enhanced accuracy of screening. The multiplexed probe-based PCR systems in one embodiment, use DNA as input material. The systems can be implemented using Real Time Polymerase Reaction (PCR), droplet-based digital PCR (ddPCR) and nanoplate-based digital PCR (dPCR). For completeness and comparison purposes, the multiplexed probe-based PCR systems can optionally use RNA as input material.

The systems identify the Mut A, Mut B, and Mut D mutations of NPM1, which account for more than 90% frameshift mutations at this locus. Therefore, in some forms, the systems include 4 probes targeting wild-type NPM1, type A NPM1 mutant (designated as Mut A), type B NPM1 mutant (designated as Mut B) and type D NPM1 mutant (designated as Mut D) for mutation screening. Typically, the probes are pooled within a single PCR reaction well and amplified sharing common PCR primers. In some forms, the systems monitor measurable residual disease (MRD) in a subject. Therefore, in some forms, the systems employ only the probes that detect wild-type (WT) and the mutant (Mut) identified in the patient. Measurable residual disease (MRD; previously termed minimal residual disease) is an independent, postdiagnosis, prognostic indicator in acute myeloid leukemia (AML) that is important for risk stratification and treatment planning, in conjunction with other well-established clinical, cytogenetic, and molecular data assessed at diagnosis.

Compositions of oligonucleotide probes specific for mutations Mut A, Mut B, or Mut D are described. The probes include Locked nucleic Acids (LNAs). The probes are specific for DNA or for RNA, and are used when either DNA or RNA is used as the input material, respectively.

### A. NPM1

The systems identify the Mut A, Mut B, and Mut D mutations of the Nucleophosmin (NPM1) gene. is a ubiquitously expressed nucleolar protein involved in ribosome biogenesis, the maintenance of genomic integrity and the regulation of the ARF-p53 tumor-suppressor pathway among multiple other functions. Mutations in the corresponding gene cause a cytoplasmic dislocation of the NPM1 protein. These mutations are unique to acute myeloid leukemia (AML), a disease characterized by clonal expansion, impaired differentiation, and the proliferation of myeloid cells in the bone marrow (Zarka, et al., Genes 2020, 11, 649; doi: 10.3390/genes11060649).

The NPM1 gene product includes 294 amino acids with a molecular weight of 37 kDa. The NPM1 gene is highly conserved between humans, rodents, chicken and fish. Human NPM1 is located on chromosome 5q35 and is composed of 12 exons with sizes ranging from 58 to 358 base pairs (bp). The regular-spliced NPM1 gene has 11 exons, encoding the 294 amino acids of the mature protein. An exemplary genomic nucleic acid sequence for human Nucleophosmin is provided in Genbank accession no. NC 000005 (region 171387116 . . . 171410900).

### 1. NPM1 Mutations

NPM1 mutations are relatively common in AML, and AML with the mutated NPM1 gene is a distinct subtype according the 2016 World Health Organization (WHO) classification, due to its specific mutational profile, immunophenotype, clinical behavior and mutual exclusiveness to other recurring genomic alterations.

NPM1 mutations are persistent throughout the course of AML and disappear with remission. This finding highlights their clinical significance and use in the monitoring of minimal or measurable residual disease (MRD) following treatment. MRD provides powerful prognostic information and is increasingly incorporated in the routine management of AML. Detectable MRD is consistently associated with an increased risk of relapse and worse long-term outcomes.

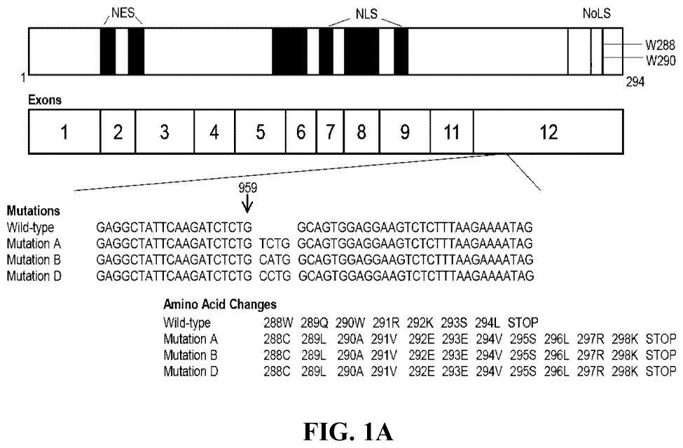

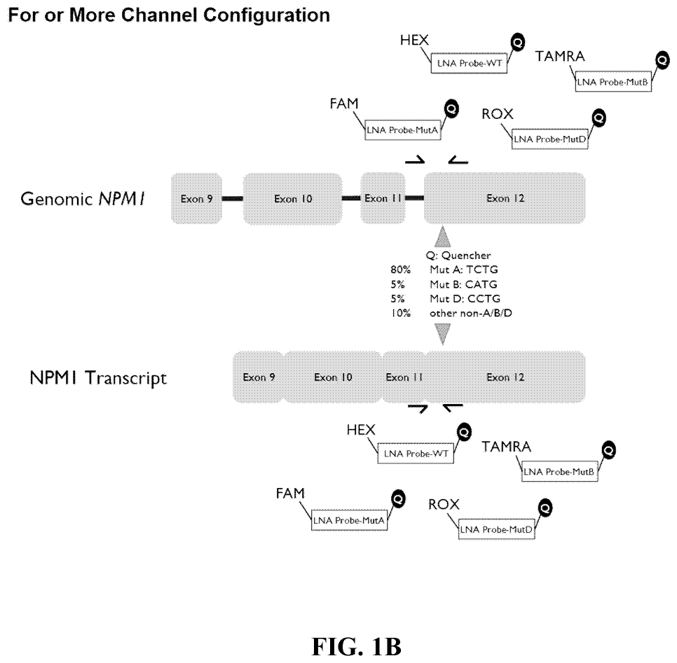

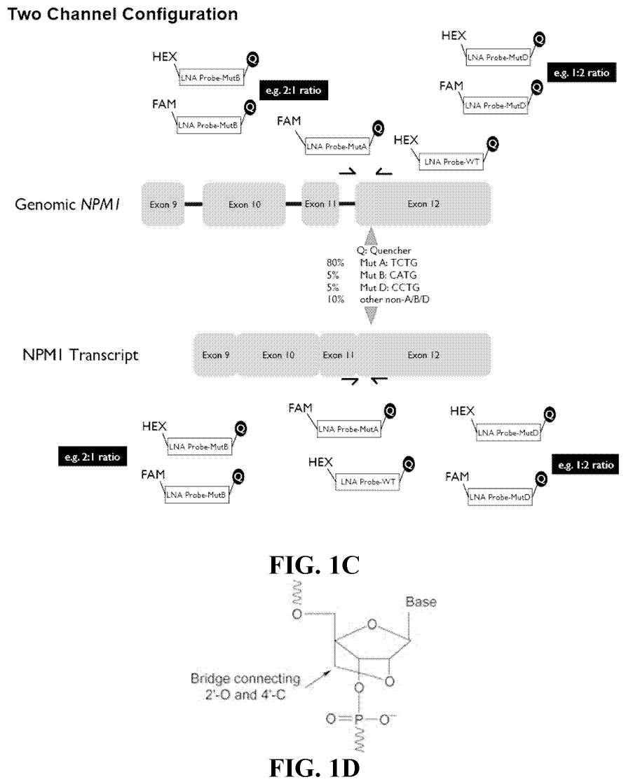

The common NPM1 mutations are found almost exclusively in exon 12, and to date have only been identified in myeloid malignancies but not in any other tumor. They often consist of 4 bp insertions or duplications between nucleotides 960 and 961. They cause the replacement of the last seven amino acids (WQWRKSL) with 11 different residues. There are three types of “common” NPM1 mutations associated with AML: A, B, and D. The positions of these NPM1 mutations are depicted in FIG. 1A.

Type A mutations, detected in 80% of cases, involve the duplication of “TCTG” (nucleotides 956-959), creating an insertion at position 960. Types B and D are the second and third most commonly occurring, followed by a few other rare mutations. All of these mutations affect the Trp-289 and Trp-290 where the NoLS resides, leading instead to a cytoplasmic localization of the protein. Cytoplasmic NPM1 (NPM1c) is only detected in AML with the NPM1-mutated gene (NPM1c), and there are no NPM1 mutations with NPM1 remaining in the nucleolus. NPM1 mutations are exclusively heterozygous, which implies that NPM1c is able to form a dimer with wild-type NPM1, recruit it to the cytoplasm and perturb its normal function (Zarka, et al., Genes 2020, 11, 649; doi: 10.3390/genes11060649).

Compositions for detecting and differentiating the wild type (WT) and mutant forms of the NPM1 gene are provided.

In some forms a nucleic acid sequence for wildtype (WT) nucleophosmin (exon 12 region) is:

(SEQ ID NO: 1)
TATGAAGTGTTGTGGTTCCTTAACCACATTTCTTTTTTTTTTTTTCCAG
GCTATTCAAGATCTCTGGCAGTGGAGGAAGTCTCTTTAAGAAAATAGTT
TAAACAATTTGTTAAAAAATTTTCCGTCTTATTTCATTTCTGTAACA.

In some forms a nucleic acid sequence for mutant A of nucleophosmin (exon 12 region) is:

(SEQ ID NO: 2)
TATGAAGTGTTGTGGTTCCTTAACCACATTTCTTTTTTTTTTTTTCCAG
GCTATTCAAGATCTCTGTCTGGCAGTGGAGGAAGTCTCTTTAAGAAAAT
AGTTTAAACAATTTGTTAAAAAATTTTCCGTCTTATTTCATTTCTGTAA
CA.

The residues associated with the mutation are highlighted in bold text and underlined.

In some forms a nucleic acid sequence for mutant B of nucleophosmin (exon 12 region) is:

(SEQ ID NO: 3)
TATGAAGTGTTGTGGTTCCTTAACCACATTTCTTTTTTTTTTTTTCCAG
GCTATTCAAGATCTCTGCATGGCAGTGGAGGAAGTCTCTTTAAGAAAAT
AGTTTAAACAATTTGTTAAAAAATTTTCCGTCTTATTTCATTTCTGTAA
CA.

The residues associated with the mutation are highlighted in bold text, and underlined.

In some forms a nucleic acid sequence for mutant D of nucleophosmin (exon 12 region) is:

(SEQ ID NO: 4)
TATGAAGTGTTGTGGTTCCTTAACCACATTTCTTTTTTTTTTTTTCCAG
GCTATTCAAGATCTCTGCCTGGCAGTGGAGGAAGTCTCTTTAAGAAAAT
AGTTTAAACAATTTGTTAAAAAATTTTCCGTCTTATTTCATTTCTGTAA
CA.

The residues associated with the mutation are highlighted in bold text, and underlined.

### B. Oligonucleotide Primers for Amplifying NPM1 DNA

Oligonucleotide primers for extending the region of the NPM1 gene associated with the most common mutations are described. In some forms the oligonucleotide primers extend a fragment of the human NPM1 gene that corresponding to all of SEQ ID Nos: 1-4.

In some forms, oligonucleotide primers extend DNA, such as genomic DNA. Exemplary oligonucleotide primers for extending the region of the NPM1 DNA associated with the most common mutations include a set of primers having the following nucleic acid sequences for forward:

(SEQ ID NO: 5)
5′ TATGAAGTGTTGTGGTTCCTTAAC 3′

and reverse:

(SEQ ID NO: 6)
5′ TGTTACAGAAATGAAATAAGACGGA 3′

oligonucleotides, respectively.

In preferred forms oligonucleotide primers are configured to produce an amplicon including the sequence of wild-type or mutant NPM1 DNA. Therefore, in some forms, oligonucleotide primers are configured to extend and amplify a fragment of DNA including the regions of mutations A, B, and D of the NPM1 gene. For example, in some forms, oligonucleotide primers are configured to produce an amplicon including the sequence of SEQ ID NO:1, or SEQ ID NO:2, or SEQ ID NO:3, or SEQ ID NO:4.

In some forms, the amplicon resulting from the extension of the DNA is between about 145 and 149 base pairs in length.

C. Oligonucleotide Primers for Amplifying NPM1 cDNA

In other forms, oligonucleotide primers extend cDNA, such as cDNA created from RT PCR of RNA. Exemplary oligonucleotide primers for extending cDNA of the region of the NPM1 associated with the most common mutations include a set of primers having the following nucleic acid sequences for forward:

(SEQ ID NO: 7)
5′ GACTGACCAAGAGGCTATTCA 3′

and reverse:

(SEQ ID NO: 8)
5′ TGTTACAGAAATGAAATAAGACGGA 3′

oligonucleotides, respectively.

Therefore, in some forms, oligonucleotide primers are configured to produce an amplicon including the sequence of wild-type or mutant NPM1 cDNA. In some forms, oligonucleotide primers are configured to extend and amplify a fragment of cDNA including the regions of mutations A, B and D of the NPM1 gene. In some forms, the amplicon resulting from the extension of the cDNA is between about 109 and 113 base pairs in length.

### D. Nucleic Acid Plasmids of NPM1 DNA

Nucleic acid plasmids including one or more sequences from the human NPM1 gene are described. In some forms the plasmids are useful as controls for systems and methods of identifying and quantifying NPM1 mutations in a sample.

In some forms, a nucleic acid sequence for wildtype (WT) nucleophosmin (exon 12 region) is:

(SEQ ID NO: 9)
CTTTATCTAGAGTTAACTCTCTGGTGGTAGAATGAAAAATAGATGTTGA
ACTATGCAAAGAGACATTTAATTTATTGATGTCTATGAAGTGTTGTGGT
TCCTTAACCACATTTCTTTTTTTTTTTTTCCAGGCTATTCAAGATCTCT
GGCAGTGGAGGAAGTCTCTTTAAGAAAATAGTTTAAACAATTTGTTAAA
AAATTTTCCGTCTTATTTCATTTCTGTAACAGTTGATATCTGGCTGTCC
TTTTTATAATGCAGAGTGAGAACTTTCCCTACCGTGTTTGATAAATGTT
GTCCAGGTTCTATTGCCAAGAATGTGTTGTCCAAAATGC.

In some forms, a nucleic acid sequence for mutant A of nucleophosmin (exon 12 region) is:

(SEQ ID NO: 10)
CTTTATCTAGAGTTAACTCTCTGGTGGTAGAATGAAAAATAGATGTTGA
ACTATGCAAAGAGACATTTAATTTATTGATGTCTATGAAGTGTTGTGGT
TCCTTAACCACATTTCTTTTTTTTTTTTTCCAGGCTATTCAAGATCTCT
GTCTGGCAGTGGAGGAAGTCTCTTTAAGAAAATAGTTTAAACAATTTGT
TAAAAAATTTTCCGTCTTATTTCATTTCTGTAACAGTTGATATCTGGCT
GTCCTTTTTATAATGCAGAGTGAGAACTTTCCCTACCGTGTTTGATAAA
TGTTGTCCAGGTTCTATTGCCAAGAATGTGTTGTCCAAAATGC.

The residues associated with the mutation are in bold text, and underlined.

In some forms, a nucleic acid sequence for mutant B of nucleophosmin (exon 12 region) is:

(SEQ ID NO: 11)
CTTTATCTAGAGTTAACTCTCTGGTGGTAGAATGAAAAATAGATGTTGA
ACTATGCAAAGAGACATTTAATTTATTGATGTCTATGAAGTGTTGTGGT
TCCTTAACCACATTTCTTTTTTTTTTTTTCCAGGCTATTCAAGATCTCT
GCATGGCAGTGGAGGAAGTCTCTTTAAGAAAATAGTTTAAACAATTTGT
TAAAAAATTTTCCGTCTTATTTCATTTCTGTAACAGTTGATATCTGGCT
GTCCTTTTTATAATGCAGAGTGAGAACTTTCCCTACCGTGTTTGATAAA
TGTTGTCCAGGTTCTATTGCCAAGAATGTGTTGTCCAAAATGC.

The residues associated with the mutation are bold text, and underlined.

In some forms, a nucleic acid sequence for mutant D of nucleophosmin (exon 12 region) is:

(SEQ ID NO: 12)
CTTTATCTAGAGTTAACTCTCTGGTGGTAGAATGAAAAATAGATGTTGA
ACTATGCAAAGAGACATTTAATTTATTGATGTCTATGAAGTGTTGTGGT
TCCTTAACCACATTTCTTTTTTTTTTTTTCCAGGCTATTCAAGATCTCT
GCCTGGCAGTGGAGGAAGTCTCTTTAAGAAAATAGTTTAAACAATTTGT
TAAAAAATTTTCCGTCTTATTTCATTTCTGTAACAGTTGATATCTGGCT
GTCCTTTTTATAATGCAGAGTGAGAACTTTCCCTACCGTGTTTGATAAA
TGTTGTCCAGGTTCTATTGCCAAGAATGTGTTGTCCAAAATGC.

The residues associated with the mutation are in bold text, and underlined.

In some forms, the nucleic acid sequences of WT or mutant A, B, or D of the NPM1 gene are combined within nucleic acid plasmids. For example, the nucleic acid plasmids can be used as controls for the identification and/or quantitation of NPM1 mutants in a sample. Therefore, in some forms, nucleic acid plasmids include any one or more of SEQ ID Nos: 1-4.

Alternatively, synthesized double-stranded DNA fragments such as gBlock DNA fragments may also be used as controls for the systems and methods as described herein.

E. Nucleic Acid Plasmids of NPM1 cDNA

In other forms, nucleic acid sequences for NPM1 WT and mutations are in the form of complementary DNA (cDNA), for example, cDNA prepared from RNA within a sample. Therefore, in some forms, a cDNA sequence for wildtype (WT) nucleophosmin (exon 12 region) cDNA is:

(SEQ ID NO: 13)
GGACAAGAATCCTTCAAGAAACAGGAAAAAACTCCTAAAACACCAAAAG
GACCTAGTTCTGTAGAAGACATTAAAGCAAAAATGCAAGCAAGTATAGA
AAAAGGTGGTTCTCTTCCCAAAGTGGAAGCCAAATTCATCAATTATGTG
AAGAATTGCTTCCGGATGACTGACCAAGAGGCTATTCAAGATCTCTGGC
AGTGGAGGAAGTCTCTTTAAGAAAATAGTTTAAACAATTTGTTAAAAAA
TTTTCCGTCTTATTTCATTTCTGTAACAGTTGATATCTGGCTGTCCTTT
TTATAATGCAGAGTGAGAACTTTCCCTACCGTGTTTGATAAATGTTGTC
CAGGTTCTATTGCCAAGAATGTGTTGTCCAAAATGC.

In some forms a nucleic acid sequence for mutant A of nucleophosmin (exon 12 region) cDNA is:

(SEQ ID NO: 14)
GGACAAGAATCCTTCAAGAAACAGGAAAAAACTCCTAAAACACCAAAAG
GACCTAGTTCTGTAGAAGACATTAAAGCAAAAATGCAAGCAAGTATAGA
AAAAGGTGGTTCTCTTCCCAAAGTGGAAGCCAAATTCATCAATTATGTG
AAGAATTGCTTCCGGATGACTGACCAAGAGGCTATTCAAGATCTCTGTC
TGGCAGTGGAGGAAGTCTCTTTAAGAAAATAGTTTAAACAATTTGTTAA
AAAATTTTCCGTCTTATTTCATTTCTGTAACAGTTGATATCTGGCTGTC
CTTTTTATAATGCAGAGTGAGAACTTTCCCTACCGTGTTTGATAAATGT
TGTCCAGGTTCTATTGCCAAGAATGTGTTGTCCAAAATGC.

The residues associated with the mutation are highlighted in bold text.

In some forms a nucleic acid sequence for mutant B of nucleophosmin (exon 12 region) cDNA is:

(SEQ ID NO: 15)
GGACAAGAATCCTTCAAGAAACAGGAAAAAACTCCTAAAACACCAAAAG
GACCTAGTTCTGTAGAAGACATTAAAGCAAAAATGCAAGCAAGTATAGA
AAAAGGTGGTTCTCTTCCCAAAGTGGAAGCCAAATTCATCAATTATGTG
AAGAATTGCTTCCGGATGACTGACCAAGAGGCTATTCAAGATCTCTGCA
TGGCAGTGGAGGAAGTCTCTTTAAGAAAATAGTTTAAACAATTTGTTAA
AAAATTTTCCGTCTTATTTCATTTCTGTAACAGTTGATATCTGGCTGTC
CTTTTTATAATGCAGAGTGAGAACTTTCCCTACCGTGTTTGATAAATGT
TGTCCAGGTTCTATTGCCAAGAATGTGTTGTCCAAAATGC.

The residues associated with the mutation are highlighted in bold text.

In some forms a nucleic acid sequence for mutant D of nucleophosmin (exon 12 region) cDNA is:

(SEQ ID NO: 16)
GGACAAGAATCCTTCAAGAAACAGGAAAAAACTCCTAAAACACCAAAAG
GACCTAGTTCTGTAGAAGACATTAAAGCAAAAATGCAAGCAAGTATAGA
AAAAGGTGGTTCTCTTCCCAAAGTGGAAGCCAAATTCATCAATTATGTG
AAGAATTGCTTCCGGATGACTGACCAAGAGGCTATTCAAGATCTCTGCC
TGGCAGTGGAGGAAGTCTCTTTAAGAAAATAGTTTAAACAATTTGTTAA
AAAATTTTCCGTCTTATTTCATTTCTGTAACAGTTGATATCTGGCTGTC
CTTTTTATAATGCAGAGTGAGAACTTTCCCTACCGTGTTTGATAAATGT
TGTCCAGGTTCTATTGCCAAGAATGTGTTGTCCAAAATGC.

The residues associated with the mutation are in bold text, and underlined.

In some forms, the nucleic acid sequences of WT or mutant A, B or D of the NPM1 gene are combined within nucleic acid plasmids to be used as controls for the identification and/or quantitation of NPM1 mutants in a sample. Therefore, in some forms, nucleic acid plasmids include any one or more of SEQ ID Nos: 13-16.

Alternatively, synthesized double-stranded DNA fragments such as gBlock DNA fragments may also be used as controls for the systems and methods as described herein.

### F. NPM1 Wild Type or Mutant Sequence Probes

Systems for identifying and quantifying NPM1 mutants use differential hybridization to detect and distinguish NPM1 polymorphisms. NPM1 DNA-binding probes including detectable labels, preferably, one or more dyes are described. Typically, the probes selectively bind to human NPM1 DNA including one of the three common mutants (A, B, or D), or to wild-type NPM1 (non-mutant) DNA. The probes are highly selective and do not non-specifically bind to NPM1 DNA. The probes are useful for identifying and quantifying NPM1 mutations in a sample. In some forms, the probes include a sequence of residues including Locked nucleic Acid (LNA) residues, and one or more dye moieties. Probes are designed to selectively hybridize to DNA amplified by PCR (amplicons) including the nucleic acid sequence of the wild-type or mutant NPM1. The probe-amplicon complex is then identified and quantified by detection of the fluorescent dye attached to the probe. Exemplary probe sequences and configurations are provided in Table 1.

### 1. Probe Sequences

Typically, probes are designed to interact with a specific sequence of residues unique to wild type, or mutant A, B or D NPM1 DNA.

An exemplary sequence for the wild-type NPM1 probe is

(SEQ ID NO: 17)
5′ACTG+CCA+G+A+GATC 3′

An exemplary sequence for the Mutant A NPM1 probe is

(SEQ ID NO: 18)
5′ TG+CCA+G+A+CA+GA 3′

An exemplary sequence for the Mutant B NPM1 probe is

(SEQ ID NO: 19)
5′ TG+CC+A+T+GC+AGA 3′

An exemplary sequence for the Mutant B NPM1 probe is

(SEQ ID NO: 20)
5′ TGCC+A+T+GC+A+GA 3′

An exemplary sequence for the Mutant D NPM1 probe is

(SEQ ID NO: 21)
5′ TGCCA+G+G+CAGA 3′

Another exemplary sequence for the wild-type NPM1 probe is

(SEQ ID NO: 22)
5′ TG+C+CA+G+A+GAT 3′

Another exemplary sequence for the Mutant A NPM1 probe is

(SEQ ID NO: 23)
5′ CT+GCCA+G+A+CA+GA 3′

Typically, the probe sequences are highly selective and do not mismatch with one another at the correct melting temperature.

### 2. Locked Nucleic Acids

In some forms, the probes include Locked Nucleic Acids (LNA). In some forms, the probe includes 2, 3, 4, 5, or 6 LNAs. Locked nucleic acids are modified RNA monomers. The “locked” part of their name comes from a methylene bridge bond linking the 2′ oxygen to the 4′ carbon of the RNA pentose ring. The bridge bond fixes the pentose ring in the 3′-endo conformation (FIG. 1D). These bases follow Watson-Crick base-pairing rules when mixed with DNA or RNA bases in an oligonucleotide.

When used in oligonucleotide probes, LNA monomers provide increased structural stability, resulting in increased hybridization melting temperature (Tm). The increase in Tm means that locked nucleic acid qPCR probes are designed with shorter lengths than standard probes, which are more effectively quenched and have a higher signal-to-noise ratio. They are, therefore, more sensitive, providing robust target detection regardless of sequence GC content. LNA probes also show greater mismatch discrimination compared to traditional qPCR probes. This improves their ability to distinguish mutations or single nucleotide polymorphisms (SNPs). (Davalieva, et al. (2014). J Virol Methods, 196:104-112). The plus sign (+) is used to designate LNA modifications in a nucleic acid sequence (see for example, Thayer, et al., Scientific Reports, 9 (3566) (2019), FIG. 1, therein). When an oligonucleotide contains standard nucleotides and LNA nucleotides in the same molecule, the term “mixmer” is sometimes invoked to more aptly describe their nature. When describing the sequence of a mixmer, one of the conventions is to use lower case letters for standard nucleotides, and upper case letters for LNA nucleotides, while another convention is to place a+symbol in front of upper case letters to represent an LNA nucleotide. For example, the primer gctGcacgTcatcgatcATCtcatgc (SEQ ID NO:24), a 26-mer with LNAs in positions 4, 9, 18, 19, and 20, can also be written as GCT+GCACG+TCATCGATC+A+T+CTCATGC (SEQ ID NO:25). By comparison, the non-LNA version of this primer is written as gctgcacgtcategatcatctcatgc (SEQ ID NO:26) (or in upper case) (Robert E. Farrell Jr. Ph.D, in RNA Methodologies (Fourth Edition), 2010). When designing LNA-containing oligonucleotides, attention is given to the location and number of LNAs. For example, a typical 18-mer should preferably contain a maximum of 7-8 LNAs; avoid stretches of more than 4-5 consecutive LNAs, which would result in very tight hybridization in that region; avoid stretches of LNAs are to be avoided close to the 3 end of an oligonucleotide, etc.

### 3. Detectable Labels

The detectable labels preferable are one or more dyes to enable detection and quantitation of probe/amplicon hybrids. In some forms, probes include at least one detectable label, preferably, a labelling dye, for example, located at the 5′ end of the probe sequence. The labelling dye is typically a fluorescent dye. In some forms, target-specific probe is labelled at one end with a fluorescent label and at the other with a quencher. In some forms, the probe further comprises an internal quencher. In some forms, fluorescent dyes attached to probes are detected to identify and quantify the NPM1 DNA in a sample. So called FRET (fluorescence resonance energy transfer) probes are based on the principle of emitting reporter and absorbing quencher in proximity. Only when there is distance between reporter and quencher, the emission can be fully detected.

In some forms, the fluorescent label is a FREEDOM™ Dye from Integrated DNA Technologies (IDT). FREEDOM™ Dyes are fluorophores with no licensing restrictions from IDT or third-party companies. Therefore, they are free to use for commercial or diagnostic applications. Freedom dyes include IDT proprietary fluorophores that are available for commonly used dye wavelengths. The numerical value in the name represents the emission wavelength of the dye when attached to an oligo.

Exemplary labelling fluorescent dyes for use in probes include 6-FAM (Fluorescein), Fluorescein dT, Cy3™, TAMRA™ dyes (e.g., 5 (6)-Carboxytetramethylrhodamine, TMR, TRITC), JOE, Cy5™, MAX, TET™, TEX615, TYE665, 6-ROX, Cy5.5™, YAKIMA YELLOW™, TEX615, TYE665, TYE705, SUN, ATTO488, ATTO532, ATTO550, ATTO565, ATTORHO101, ATTO590, ATTO633, ATTO647N, HEX, and Alexa Fluor dyes.

Probes are preferably designed such that a quencher and fluorophore always remain in close proximity if the specific target is not present, and be widely separated if it is present. Observation of a fluorescent signal thus indicates presence of target, and lack of a fluorescent signal indicates absence of target.

In some forms, the probes also include at least one dark quenching, non-fluorescent dye, for example, located at the 3′ end of the probe sequence. Dark quenchers are dyes with no native fluorescence and enable multiplexing (when two or more reporter-quencher probes are used together). Non-fluorescent dark quenchers can quench the fluorescence of dyes in bioassays, reducing background.

Exemplary quenching dyes for use in probes include IOWA BLACK® RQ quenchers, IOWA BLACK® FQ quenchers, IAbRQSp, Dabsyl (dimethylaminoazobenzenesulfonic acid), Black Hole Quenchers, Qxl quenchers, IRDye QC-1, and internal ZEN® Quencher.

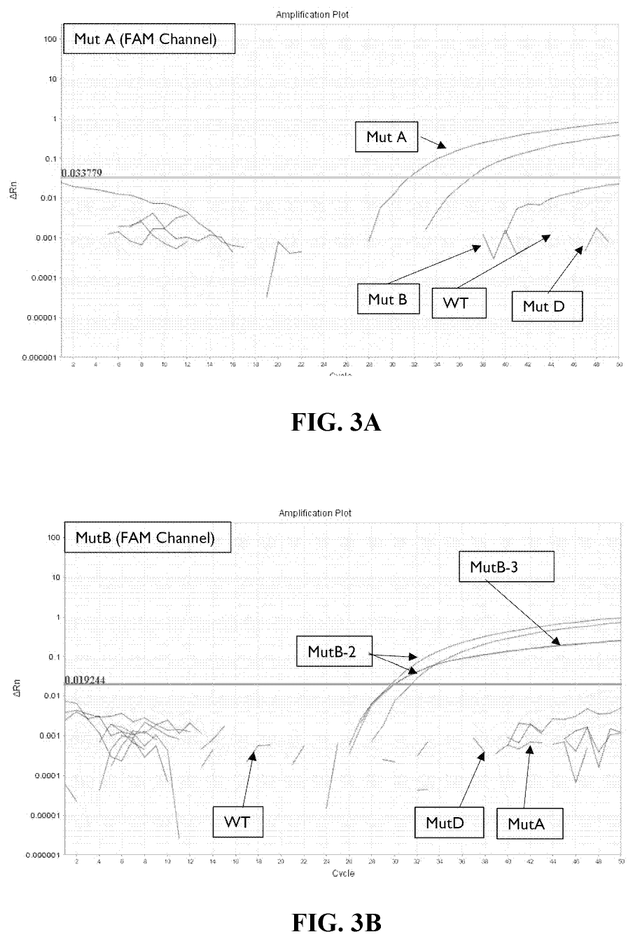

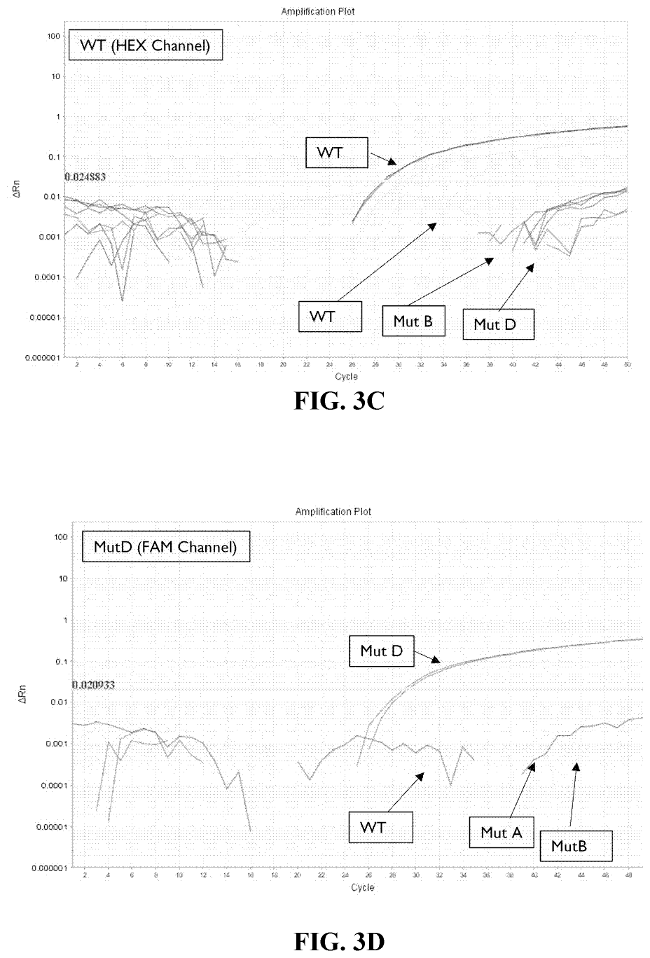

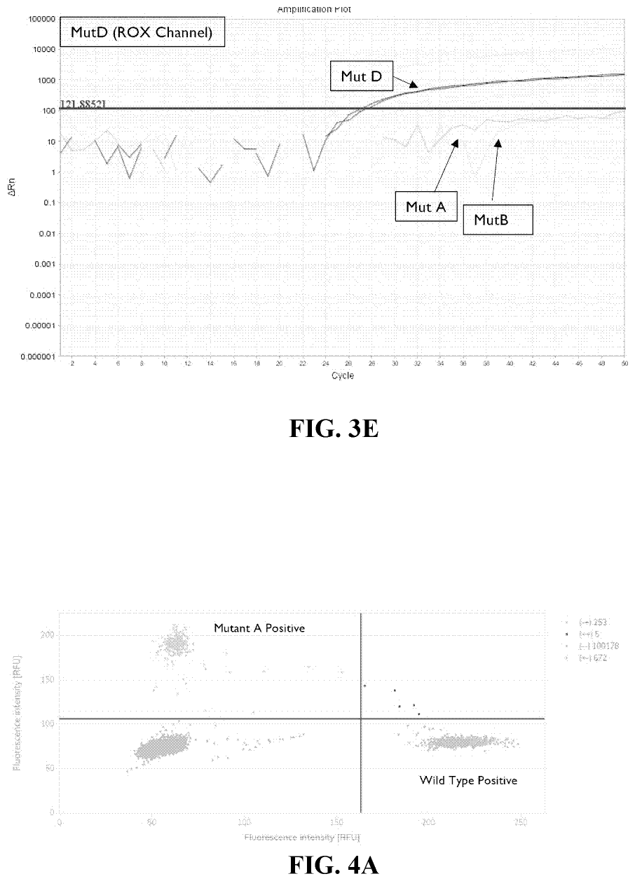

Methods of labeling probed are known are known. For example, TaqMan probes are labeled with two fluorescent dyes that emit at different wavelengths (FIG. 3.4). The probe sequence is intended to hybridize specifically in the DNA target region of interest between the two PCR primers. Typically the probe is designed to have a slightly higher annealing temperature compared to the PCR primers so that the probe will be hybridized when extension (polymerization) of the primers begins. A minor groove binder is sometimes used near the 3′-end of TaqMan probes to enable the use of shorter sequences that have higher annealing temperatures than would be expected for sequences of equivalent length. The “reporter” (R) dye is attached at the 5′-end of the probe sequence while the “quencher” (Q) dye is synthesized on the 3′-end. A popular combination of dyes is FAM or VIC for the reporter dye and TAMRA for the quencher dye. When the probe is intact and the reporter dye is in close proximity to the quencher dye, little to no fluorescence will result because of suppression of the reporter fluorescence due to an energy transfer between the two dyes.

During polymerization, strand synthesis will begin to displace any TaqMan probes that have hybridized to the target sequence. The Taq DNA polymerase used has a 5′-exonuclease activity and therefore will begin to chew away at any sequences in its path (i.e., those probes that have annealed to the target sequence). When the reporter dye molecule is released from the probe and is no longer in close proximity to the quencher dye, it can begin to fluoresce. As a result, the fluorescence signal of the reporter fluorochrome will become detectable and further increases during the consecutive PCR cycles because of the progressive accumulation of free reporter fluorochromes.

### III. Methods of Use

Methods for the reliable molecular monitoring of NPM1 mutations have been established. The methods enable pre-emptive therapy essential for understanding AML disease status, informing therapeutic options, monitoring therapeutic efficacy and improving patient outcome. The methods simultaneously screen and quantify samples having the major NPM1 mutant subtypes.

In some forms, methods for detecting and measuring a presence, absence and/or level of mutation of the NPM1 gene in a sample include one or more of the following steps:
(a) hybridizing labelled sequence probes to the NPM1 gene in a sample to form probe-nucleic acid conjugates.
(b) subjecting the probe-nucleic acid conjugates to an amplification step. Amplifying typically includes polymerase chain reaction (PCR). PCR is typically carried out using Real Time-PCR, and/or digital PCR, or digital droplet PCR systems.
(c) detecting the an amplification product. Detection is typically through measuring the level of one or more of the fluorescent labels attached to the probes.

The methods also optionally include one or more steps for
(d) Recording the presence, absence and/or level of NPM1 mutations in the sample. The identification of a mutation, and quantitation thereof can be carried out according to the method employed for detection in step (c).
   The methods can, at all steps, employ the use of one or more controls.

In some forms, controls are samples including DNA obtained from a subject known to have a mutant NPM1 gene or a wildtype NPM1 gene. In particular forms, the controls include nucleic acid plasmids including one or more of SEQ ID NOs: 1-4, or SEQ ID NOs: 9-16.

### A. Hybridizing Labelled Sequence Probes to the NPM1 Gene in a Sample to Form Probe-Nucleic Acid Conjugates

The methods include one or more steps of hybridizing labelled sequence probes to DNA corresponding to a fragment of the NPM1 gene within a sample. Therefore, the methods include providing a sample for screening. Exemplary samples used in the methods include samples of DNA or RNA obtained from a subject, or from a synthetic source. DNA samples include purified genomic DNA, and fragments thereof, or cDNA that is prepared from RNA obtained from a subject. Synthetic DNA samples include plasmids, cosmids, as well as single or double-stranded DNA, such as from within a library.

The hybridizing includes the formation of a probe-NPM1 nucleic acid conjugate. In some forms, formation of a probe-NPM1 nucleic acid conjugate is carried out in a single reaction vessel, for example, for simultaneous screening of all three mutants together, or for screening of one or two mutants. In some forms, the methods perform amplification of NPM1 DNA within the sample and probe binding within the same reaction. Therefore, in some forms, the methods include one or more steps of
(i) combining in a reaction mixture under primer extension conditions a set of primers with the sample. A set of primers typically includes two primers (one reverse and one forward), to amplify the NPM1 DNA.

The primers include sequences complementary to the NPM1 gene and are configured to produce an extension product comprising all or part of the NPM1 gene. Exemplary primer sets for DNA samples are set forth in SEQ ID NOs: 5-6. Exemplary primer sets for cDNA samples are set forth in SEQ ID NOs: 7-8.

In some forms, the methods include one or more steps of
(ii) adding to the reaction mixture one or more labelled sequence probes.
   Each of the probes typically includes Locked nucleic Acid (LNA) having a sequence complementary to the NPM1 gene, or a mutant of the NPM1 gene; a reporter dye, and a quencher. Typically, the sequence complementary to the NPM1 gene anneals to the extension product at a predetermined location.

### B. Amplifying Probe-Nucleic Acid Conjugates.

The methods include one or more steps for Amplifying the Probe-Nucleic Acid Conjugates. The step of amplifying the conjugates can be done by means known in the art. Amplification typically includes polymerase chain reaction (PCR) using Real Time-PCR, and/or digital PCR, or digital droplet PCR systems. In some forms, conjugation of probes and amplification according to the methods is carried out using a single reaction mixture containing all primers and probes as well as sample DNA and reagents required for PCR.

In an exemplary form, the methods screen mutations using 4 probes targeting wild-type NPM1, type A NPM1 mutant, type B NPM1 mutant and type D NPM1 mutant. The methods pool the probes in a single PCR reaction well and amplify the DNA/probe conjugates using PCR sharing common PCR primers.

The methods are implemented using PCR instruments known in the art. Exemplary PCR instruments that can be used to implement the methods include the following instruments: Step One Plus (SOP) Real Time PCR system, Bio-Rad QX200 ddPCR system, Qiagen QIACUITY® dPCR system, Roche LC480 II Real Time PCR instruments and ThermoFisher Quantstudio 3D dPCR system.

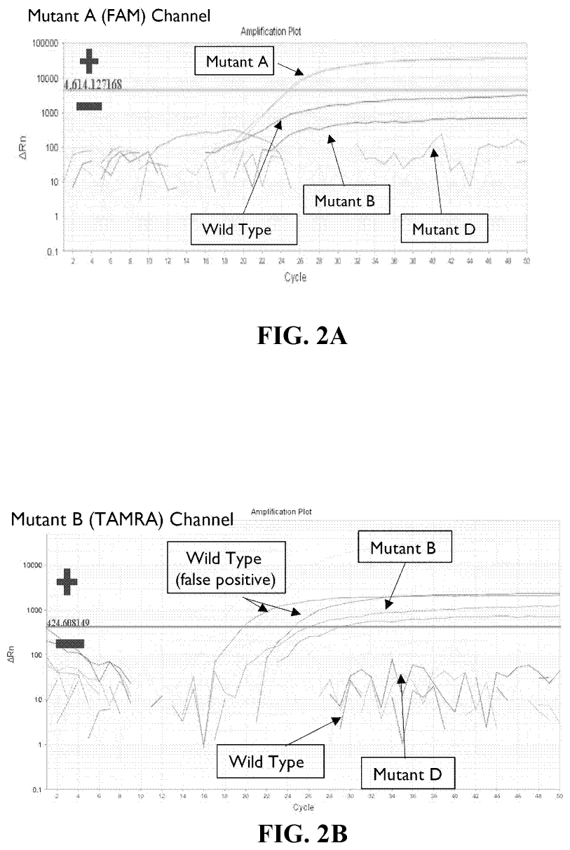

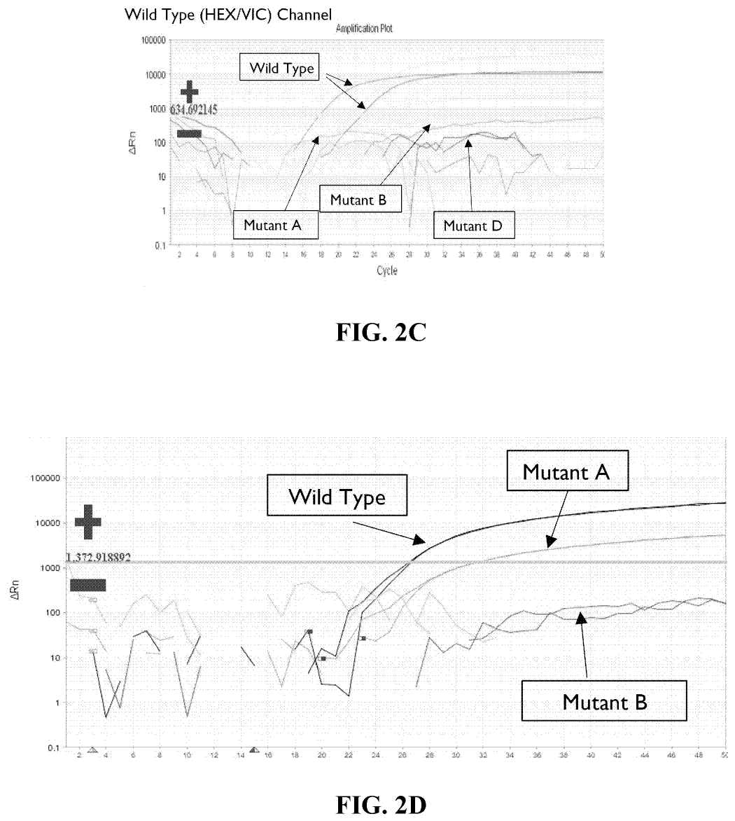

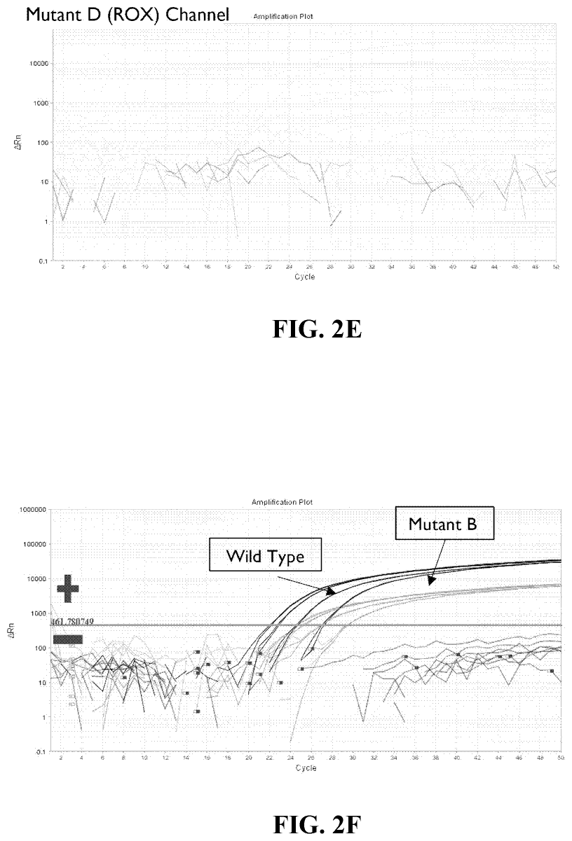

In some forms, each sample is tested and analyzed as technical duplicates. An exemplary detection of different mutations carried out using Real Time PCR according to the methods is set forth in FIGS. 2A-2F.

In an exemplary method, optimal PCR conditions included 800 nM of primers with 400 nM for each of the four probes in a final volume of either 40 μL reaction for dPCR or 20 μL reaction on ddPCR and Real Time PCR. An exemplary PCR condition uses oligonucleotide primers of SEQ ID NOs: 5 and 6, and includes initial heating to 95° C. for 20 seconds, followed by 40 cycles of 95° C. for 1 second to 30 seconds and 60° C. for 5 seconds to 1 minute, depending on the PCR machine.

### C. Detecting the Detectable Labels.

The methods include one or more steps for detecting the results of the amplification step typically by detecting the detectable label, for example, fluorescence, where the detectable label is a fluorescent dye. The step of detecting is typically through measuring the level of one or more of the fluorescent labels attached to the probes. Therefore, in some forms the methods measure and/or record the presence and quantity of fluorescent labels that were attached to probe conjugates. In some forms the methods are implemented in Real Time PCR systems and the measurement and/or recording of fluorescent labels is carried out and displayed in Real Time, for example, in the form of a visual display implemented on a computer.

In some forms one type of DNA template is loaded into each well, e.g., either plasmid control of Mut A/B/D/WT NPM1 or patient DNA which patient DNA was previously known to carry both Mut and WT NPM1. In other forms each 4-plex well is loaded with different types of templates, and correct positive signals are obtained in Mut A and WT channel with no false positive signals in well loaded with Mut B and Mut D plasmid controls. In an exemplary method, when Mut A template is loaded, a distinct amplification is observed in the Mut A FAM channel marked by two sigmoid curves tightly overlapped and intersected with the green horizontal threshold, for example, as set forth in FIG. 2A. In preferred forms, the LNA probe for Mut A provides the desired specificity with no binding to any of the other mutants or WT NPM1 and other channels do not show signals higher than the cutoff threshold, indicating the. In preferred forms, the LNA probe for WT NPM1 shows the desired positive signals only detected in the WT fluorescent channel, for example, as set forth in FIGS. 2C-2D.

As discussed above, in some forms the methods include controls. Exemplary controls include DNA samples including positive and negative controls. In preferred forms, no false positive signal is detected in control wells with no DNA template, or spiked with incorrect types of plasmids, and positive signals are detected in positive control wells for each of wild-type, mutant A, mutant B and mutant D NPM1.

D. Recording the Presence, Absence and/or Level of NPM1 Mutations in the Sample.

The methods optionally include one or more steps for recording the identification and quantitation of an NPM1 mutation. Typically, the identification is carried out according to the method employed for detection in step (c). Typically, the methods enable detection and quantitation of a NPM1 mutant based on the presence of as few as 1000 copies of the NPM1 DNA or less than 100 copies of the NPM1 DNA, for example, 100 copies or less, 10 copies or less, 5 copies or less, or one copy. Typically, the limit of detection of the methods is dependent upon the method of PCR amplification platform employed. For example, in some forms the methods enable detection and quantitation of a NPM1 wild type or mutant DNA based on the presence of as few as 100 copies, or less than 100 copies of the NPM1 DNA when Real Time PCR is employed. In other forms the methods enable detection and quantitation of a NPM1 wild type or mutant DNA based on the presence of as few as 10 copies, or less than 10 copies of the NPM1 DNA when digital PCR is employed.

The described methods for determining the presence and quantity of NPM1 mutations are useful for diagnosing diseases and disorders associated with NPM1 mutations in a subject. Therefore, in some forms, the methods include one or more steps of diagnosing and/or monitoring the state of a disease or disorder in a subject. In some forms the methods identify and measure measurable residual disease (MRD) in a subject. An exemplary disease in acute myeloid leukemia (AML).

### E. Selecting Samples

In some forms, the methods include one or more steps of identifying a subject in need of monitoring for NPM1 mutations and obtaining a suitable sample from the subject. Typically, the subject is one with leukemia.

In some forms, the methods characterize tumors and/or characterize the tumor microenvironment in a subject with leukemia. For example, in some forms, the methods identify tumor cells and/or tumor infiltrating cells or tumor associated cells as having NPM1 mutations.

Methods for assessing the anti-cancer efficacy of chemotherapeutic agents or other procedures are also disclosed. In some forms the methods include steps of assessing disease state in a subject prior to and following measuring NPM1 gene mutations in a sample from the subject. For example, in some forms, blood samples from cancer patients are characterized prior to and following the described methods for detecting and quantifying NPM1 mutations, in order to monitor changes in disease as phenotypic and/or genetic changes associated with a therapeutic regimen.

Typically, the subject is a subject that has blood cancer, and that is currently or has previously been administered treatment for the cancer, and wherein the anti-cancer response achieved following the administration of the treatment is identified by the methods.

The methods are particularly effective for monitoring acute myeloid leukemia (AML) characterized by mutations in the NPM1 gene. In preferred forms, the compositions and methods are effective in diagnosing or monitoring one or more types of leukemia.

All of the methods described may include one or more steps of identifying a subject and/or obtaining a biological sample from a subject. Suitable methods of obtaining biological samples from a subject and obtaining purified RNA or DNA from the biological sample are known in the art. In some forms, the methods include taking multiple samples from a subject over a period of time. For example, in some forms a multiplicity of samples is taken from the same subject over a predetermined period of time, for example, according to a treatment regimen. In an exemplary form, a multiplicity of samples is taken from the same subject to monitor the presence or absence or quantity of mutations in the NPM1 gene following one or more of cancer diagnosis, cancer treatment, or assessment of cancer remission. In some forms, a sample is obtained from a dead subject. In some forms, a sample is obtained to measure residual disease in the subject, for example, following hematopoietic stem cell therapy (HSCT). If a sample includes RNA, the methods include one or more steps for reverse-transcription to prepare purified cDNA.

### IV. Kits

Kits are also disclosed. Kits for detecting and measuring a presence, absence and/or level of mutation of the NPM1 gene in a sample include one or more reagents including (i) oligonucleotide primers; and (ii) one or more labelled sequence probes. The kits optionally further include (iii) instructions for a method of detecting and measuring a presence, absence and/or level of mutation of the NPM1 gene in a sample. Typically, the primers include nucleic acid sequences complementary to the NPM1 gene and are configured to produce an extension product including all or part of the NPM1 gene. Typically, the probes include a nucleic acid sequence complementary to wild-type NPM1 or a mutant of the NPM1 gene, which anneals to the extension product at a predetermined location; and a reporter dye and a quencher dye. Preferably the probes include Locked nucleic Acid (LNA). The kits can include, for example, reagents supplied alone (e.g., lyophilized), or in a pre-assembled admixture. The active agent(s) can be in an amount suitable for immediate use with a single or multiple sample(s) by admixture, or in a stock that should be diluted prior to administration. In some forms, the kit includes a supply of buffers and solutes, packaged in individual vials. The kit can also include devices for obtaining a biological sample from a subject, for example, syringes, and/or for purification of DNA from the biological sample, or for preparation of cDNA from an RNA sample. The kits can include printed instructions for administering the compound in a method as described above.

The disclosed compositions and methods can be further understood through the following examples.

### EXAMPLES

### Example 1: Screening for Mutations in NPM1 Gene Using Real Time PCR Methods

### Specificity Test for Mutations Screening Purposes

The 4-plex and 2-plex PCR system was tested on Real Time PCR (SOP), ddPCR (QX200) and dPCR (QIAcuity One 4-Plex system) as a proof-of-principle.

Mut A, Mut B and Mut D account for more than 90% frameshift mutations at this locus (FIG. 1A), the methods enable identification of the majority of commonly reported NPM1 mutations.

The mutations and assay design of the methods are set forth in FIGS. 1A-1C.

### Primer Design

The probes are specific to MutA, Mut B and MutD, with no false positivity being detected to these 3 types of mutations. Two sets of PCR primers were designed, one set for DNA as input material and another set for RNA. Four LNA probes targeting NPM1 are designed to specifically differentiate WT NPM1 from Mut A, Mut B or Mut D. The primer and probe sequences designed for the method are set forth in Table 1, with melting temperatures (Tm) of perfect match and mismatches listed.

During PCR, all mutant A, B and D probes are employed in pairs, however each mutant probe is labelled with a different reporter dye at 5′ end and different quencher dye at 3′ end.

TABLE 1
PCR Primer and Probe sequences labelled with different combinations of
reporter and quencher dyes

| | | Tm | | | | | | |
|---|---|---|---|---|---|---|---|---|
| | | (° C.) | Amplicon Size | | | | | |
| NPM1 DNA PCR primer | | | | | | | | |
| Forward | TATGAAGTGTT | 61.5 | 145 bp (WT) & | | | | | |
| | GTGGTTCCTTA | | 149 bp (Mut | | | | | |
| | AC | | A/B/D) | | | | | |
| Reverse | TGTTACAGAAA | 60.8 | | | | | | |
| | TGAAATAAGAC | | | | | | | |
| | GGA | | | | | | | |
| NPM1 RNA PCR Primer | | | | | | | | |
| Forward | GACTGACCAAG | 47.6 | 109 bp (WT) & | | | | | |
| | AGGCTATTCA | | 113 bp (Mut | | | | | |
| Reverse | TGTTACAGAAA | 61.0 | A/B/D) | | | | | |
| | TGAAATAAGAC | | | | | | | |
| | GGA | | | | | | | |
| ABL RNA PCR Primer | | | | | | | | |
| Forward | TTCTTGGTGCGT | 55.0 | 112 bp | | | | | |
| | GAGAGTG | | | | | | | |
| Reverse | CGTAGAGCTTG | 57.4 | | | | | | |
| | CCATCAGAA | | | | | | | |
| | Seq | | | | | | | |
| | (Highlighted | | Mismatch Tm | | | | | |
| Probe | in bold: Best | Tm | to other types | *Dye | *Dye | *Dye | *Dye | *Dye |
| Name | Sequence) | (° C.) | of mutant | Comb 1 | Comb 2 | Comb 3 | Comb 4 | Comb 5 |
| NPM1- | **ACTG+CCA+ | 65.8 | MutA: <56° C./ | HEX+ | | | | |
| WT | G+A+GATC | | MutB: <56° C./ | BHQ1 | | | | |
| | | | MutD: <56° C. | | | | | |
| NPM1- | **TG+CCA+G+A | 66.4 | WT: <56° C./ | FAM+ | | | | |
| MutA | +CA+GA | | MutB: 30.6° C/ | BHQ1 | | | | |
| | | | MutD: 50° C. | | | | | |
| NPM1- | TG+CC+A+T+GC | 65.9 | WT: <56° C./ | 56- | | | | |
| MutB | A+GA | | MutA: 26° C./ | TAMRA | | | | |
| | | | MutD: 48° C. | + BHQ2 | | | | |
| NPM1- | **TGCCA+G+G+ | 64.3 | WT: <56° C./ | FAM+ | HEX+ | 6- | TEX6 | TYE6 |
| MutD | CAGA | | MutA: 56.9° C/ | BHQ1 | BHQ1 | ROX+ | 15+ | 65+ |
| | | | MutB: 49° C. | | | BHQ2 | BHQ2 | BHQ3 |
| NPM1- | **TG+CC+A+T+ | 64.9 | WT: <56° C./ | FAM+ | HEX+ | ATTO | | |
| MutB-2 | GC+AGA | | MutA: 25° C./ | BHQ1 | BHQ1 | 550+ | | |
| | | | MutD: 47° C. | | | BHQ2 | | |
| NPM1- | TGCC+A+T+GC | 64.8 | WT: <56° C./ | FAM+ | HEX+ | ATTO | | |
| MutB-3 | +A+GA | | MutA: 26.7° C./ | BHQ1 | BHQ1 | 550+ | | |
| | | | MutD: 47.6° C. | | | BHQ2 | | |
| NPM1- | CT+GCCA+G+A+ | 65.4 | WT: <56° C./ | FAM+ | | | | |
| MutA-2 | CA+GA | | MutB: 34° C./ | BHQ1 | | | | |
| | | | MutD: 50.8° C. | | | | | |
| NPM1- | TG+C+CA+G+A+ | 66.0 | MutA: <49° C./ | HEX+ | | | | |
| WT-2 | GAT | | MutB: <49° C./ | BHQ1 | | | | |
| | | | MutD: <52° C. | | | | | |
| ABL | **AGGGAGGGTGT | 60.7 | Not applicable | | | | | |
| | ACCATTACAGGATC | | HEX + BHQ1 | | | | | |
| | A | | | | | | | |

*Dye combinations (Reporter+ Quencher)
**preferred embodiment

The 4-plex PCR probes used for the current testing were labelled with 4 different fluorescent dyes, FAM, TAMRA, ROX and HEX, targeting NPM1 mut A, B, D, and wild-type NPM1 respectively.

### Controls

In addition to the synthetic plasmid controls used for testing, four AML patients, two with NPM1 type A mutant (Patient A) and two with type B mutant (Patient B), were used to test the platform. These patients were previously genotyped using next generation sequencing done in other laboratories. However, DNA from a patient with Mut D was not available for testing due to its rarity and thus synthetic DNA of Mut D was used as surrogate. For the control experiment, synthetic mutant plasmid controls and WT plasmid were included.

Four plasmid vectors pUCIDT-AMP are cloned with portion of intron 11-12 and exon 12 of genomic WT, or Mut A, Mut B or Mut D NPM1. These four plasmids or gBlock fragment controls serve as positive controls for corresponding genotypes when screening mutations at DNA level. They also serve positive control for subsequent MRD application by dPCR and ddPCR and serial diluted standards for Real Time PCR. The insert sequences are provided in Table 2. Similarly, another four controls are cloned with cDNA of exon 9-12 of NPM1 which serve as synthetic controls for RNA samples. For PCR of RNA samples, the RNA samples are first reverse transcribed into cDNA prior to actual PCR.

### Input Nucleic Acids

In addition to the synthetic plasmid controls used for testing, four AML patients, two with NPM1 type A mutant (Patient A) and two with type B mutant (Patient B), were used to test the platform. These patients were previously genotyped using next generation sequencing done in other laboratories. However, DNA from a patient with Mut D was not available for testing due to its rarity and thus synthetic DNA of Mut D was used as surrogate. For the control experiment, synthetic mutant plasmid controls and WT plasmid were included.

### PCR

For mutation screening, 4 probes targeting wild-type NPM1, type A NPM1 mutant (designated as Mut A), type B NPM1 mutant (designated as Mut B) and type D NPM1 mutant (designated as Mut D) were pooled in a single PCR reaction well and amplified sharing common PCR primers.

The methods were tested on the following instruments: Step One Plus (SOP) Real Time PCR system, Bio-Rad QX200 ddPCR system and Qiagen Qiacuity dPCR system. The system is compatible with other Real Time PCR instruments (e.g., Roche LC480 II) or dPCR instruments (e.g., ThermoFisher Quantstudio 3D dPCR system).

Each sample was tested and analyzed as technical duplicates. Detection of different mutations was carried out using Real Time PCR, as set forth in FIGS. 2A-2F.

### Results

Upon several rounds of optimization, the most optimal PCR condition comprised 800 nM of primers with 400 nM for each of the four probes in a final volume of either 40 μL reaction for dPCR or 20 μL reaction on Real Time PCR.

PCR condition involved initial heating of 95° C. (20 seconds) followed by 40 cycles of 95° C. (15 seconds) and 60° C. (30 seconds). During first round of testing, only one type of DNA template was loaded into each well, either plasmid control of Mut A/B/D/WT NPM1 or patient DNA which patient DNA was previously known to carry both Mut and WT NPM1.

In each 4-plex well loaded with different types of templates, correct positive signals were obtained in Mut A and WT channel with no false positive signals in wells loaded with Mut B and Mut D plasmid controls; when Mut A template was loaded, a distinct amplification was observed in the Mut A FAM channel marked by two sigmoid curves tightly overlapped and intersected with the green horizontal threshold (FIG. 2A). Other channels did not show signals higher than the cutoff threshold, indicating the LNA probe for Mut A provided the desired specificity with no binding to any of the other mutants or WT NPM1. Similarly, LNA probe for WT NPM1 showed the desired positive signals only detected in WT HEX channel (FIGS. 2C-2D). When Mut B was loaded, the signal of Mut B was weaker than expected and a strongly false positive signal was observed, suggesting this probe (NPM1-MutB) should be replaced by NPM1-MutB-2 or NPM1-MutB-3 (FIG. 2B). The Mut B probe showed cross reactivity with other types of plasmids hence false positive signals in the TAMRA channel.

To further test the system with new Mut B probes and under a complex template loading situation, duplex PCR was performed with WT probe labelled with HEX and mutant probe labelled with FAM. In each well WT probe was mixed with one type of mutant probe and was loaded with either 100,000 copies of WT or mutant plasmid controls. Additional wells spiked with the incorrect mutant plasmid was done to test for false positivity. (FIGS. 3A-3E). For WT and Mut A probes, each performance was identical to previous runs with non-false positive signals when spiked with a different mutant plasmid. For the new Mut B probes, optimal signal intensity was observed from both probes with no false positivity. For Mut D probe, both signal from FAM and ROX channel was recovered, and no false positivity recorded. All of these data indicated all probes were optimal for genotype screening.

### Example 2: Screening for Mutations in NPM1 Gene Using Digital PCR (dPCR) Methods

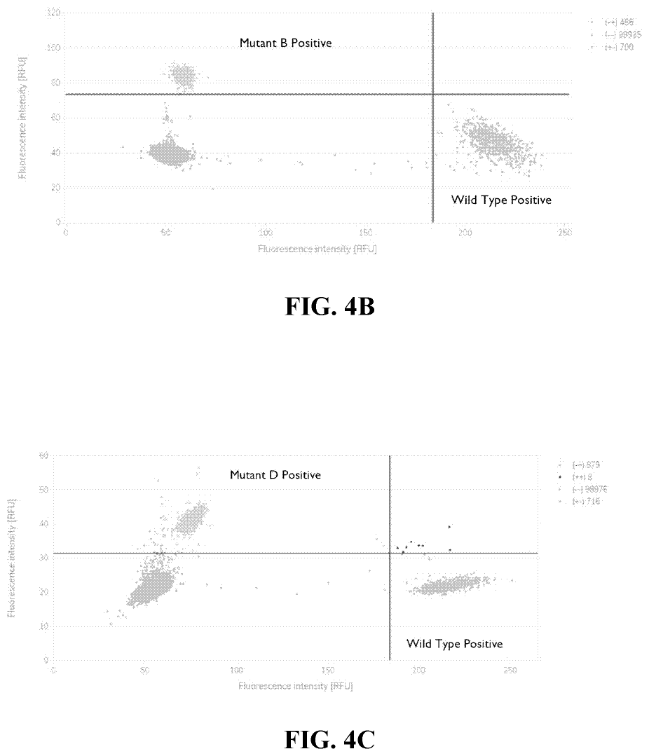

For testing of the 4-plex system in QIACUITY® dPCR system (QIAGEN), the same PCR condition was used except that the reaction volume was increased to 40 μL to be compatible with the 26k nanoplate of the QIACUITY® system. dPCR is more sensitive and allows amplification of individual template DNA by partitioning. The data confirmed this capability in detecting all three mutants and differentiating them from the WT NPM1. In the 2-dimensional (2-D) scatter plot showing collective data of all samples, positive signals were clustered and were distinct from the negative signals; Collective data of all wells loaded with same type of templates indicated desired probe specificity except Mut D showed an overall weaker signal intensity which need further optimization (FIGS. 4A-4C).

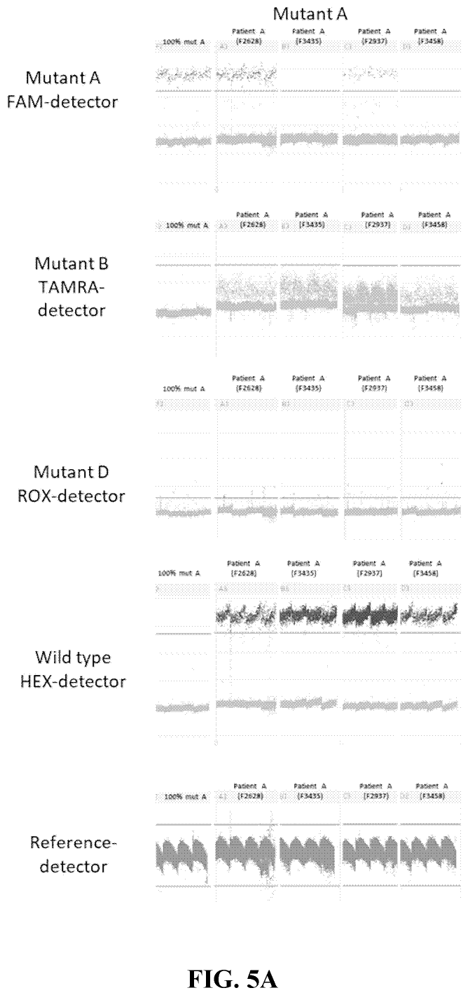

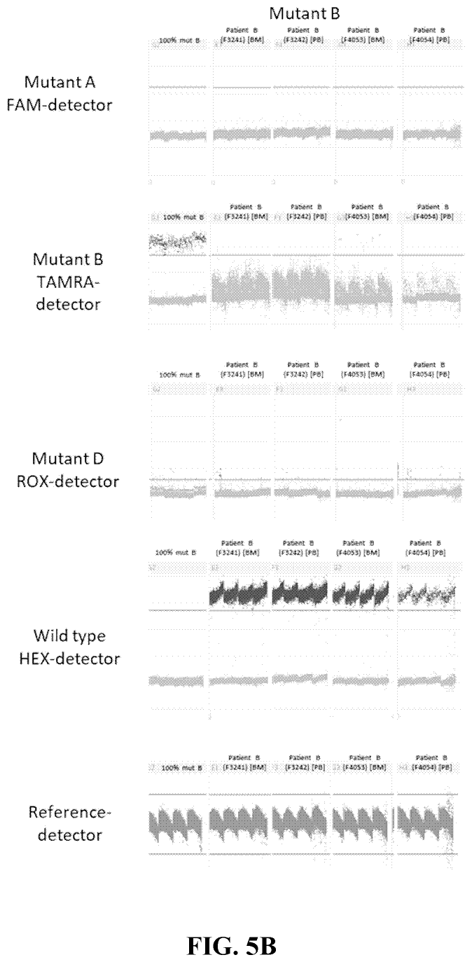

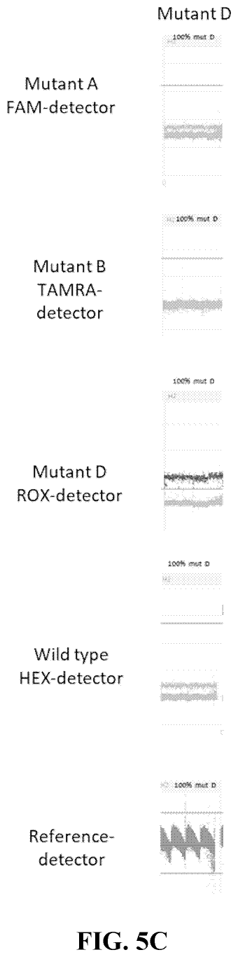

When studying individual wells loaded which different types of mutant plasmid spiked into a constant copy number of 20000 wild type NPM1, the 1-D scatter plot showed distinct and specific positive signals of mutants on the corresponding fluorescent detector channels (FIG. 5). Results of individual wells indicated Mut A, Mut D and WT all yielded highly specific signals when loaded with the correct types of template plasmid controls or patient gDNA. However, Mut B probe needs to be replaced by MutB-2 or MutB-3 due the significant noise observed in both wells loaded with mismatched patient DNA (Patient A).

The overall intensity of WT and Mut A was sufficiently high [Relative Fluorescence Unit (RFU)>150] and DNA of patient with known Mut A (Patient A) showed intensity comparable to plasmid controls. Moreover, non-specific signals were not observed in wells containing Mut B and D synthetic controls or DNA of patients with Mut B (Patient B) as desired.

The signal intensity of Mut B and D was considerably low but Mut B specific signals was only observed in wells with Mut B plasmid or patient bone marrow DNA with known Mut B (F4053). There were no positive events detected in F3241 and F3242 that were paired bone marrow (BM) and peripheral blood (PB) respectively from a patient with known Mut B. This patient underwent a series of treatment and external laboratory PCR results showed only a normalized mutation burden of 0.14% in the bone marrow. This was expected given the lower sensitivity of the peripheral blood compared to the bone marrow. However, the background noise of Mut B and D requires further optimization with LNA probe modifications and dye selection to improve signal intensity and non-specific amplification.

In contrast to the Real Time PCR where Mut D failed to amplify, dPCR showed significantly better results. In FIGS. 2A-2F and FIGS. 3A-3E, four discrete clusters of positive events for all three mutants and WT NPM1 were observed in both the 2-D and 1-D scatter plots. It also indicated that the probe for Mut D could detect the presence of Mut D plasmid controls. The probe for Mut A and WT showed the desired specificity but the probe for Mut B showed significant noise events attributed to non-specificity. The specificity of Mut D probe could not be concluded at this juncture due to the low signal intensity of Mut D (RFU<50). The specificity of this probe should be determined when a stronger positive signal is achieved using a brighter fluorescent dye with same emission wavelength but higher quantum yield in comparison with ROX dye currently used to label probe D.

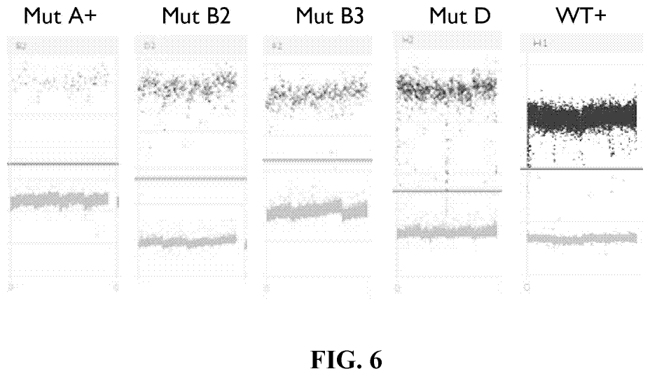

Using new probes Mut B2, B3 and Mut D labelled with FAM, the probe specificity was tested in QIACUITY® system, and optimal specificity was observed (see FIG. 6). The probes showed desired specificity, despite mild PCR inefficiency marked by the rain (weak signal) in Mut D and WT channel when Mut D and WT plasmids were loaded, respectively. An additional WT probe was also designed. Similarly, an extra Mut A2 probe was also designed.

### Example 3: Screening for Mutations in NPM1 Gene Using Digital Droplet PCR (ddPCR)

The originally designed Mut A probe was confirmed as being optimal in both the SOP and QIACUITY® system. Since originally designed Mut A probe were test optimally in both SOP and Qiacuity system and Mut B probe required replacement with new probe design when tested on both systems, Mut A and Mut B probe was no longer tested when proceeding to QX200 ddPCR system. The Mut B2, B3 and Mut D probes labelled with both FAM dye were tested on QX200 Digital Droplet PCR system duplex with WT labelled with HEX.

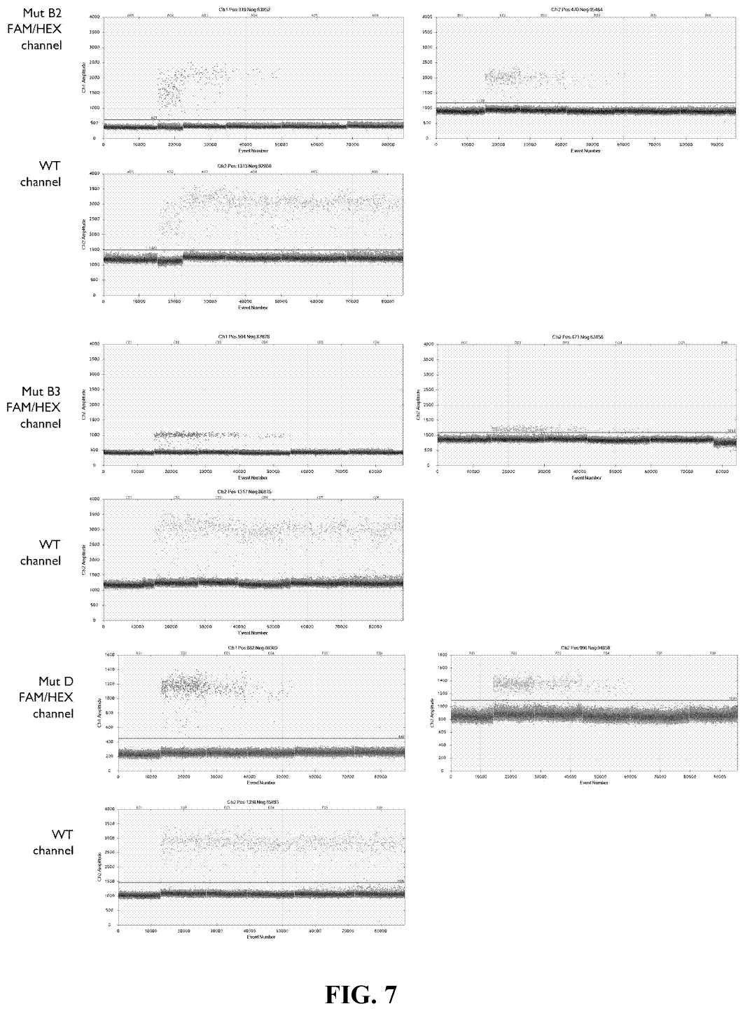

To mimic human genomic DNA, each well was loaded with a constant 20,000 copies of WT plasmid and variable copies of mutant controls or spiked with mismatch mutant plasmids. Mutant specific signals were observed in each FAM or HEX channel when loaded with Mut B or D plasmid even under presence of abundant WT plasmid. No false positive signal was detected in wells with no DNA template (NTC) or spiked with incorrect types of plasmids (FIG. 7). Therefore, as desired, correct specificity of the Mut B2, Mut B3 and Mut D probes was observed, despite raindrops (weak) signaling being observed. To counter this, optimization on probe concentration on the QX200 was necessary.

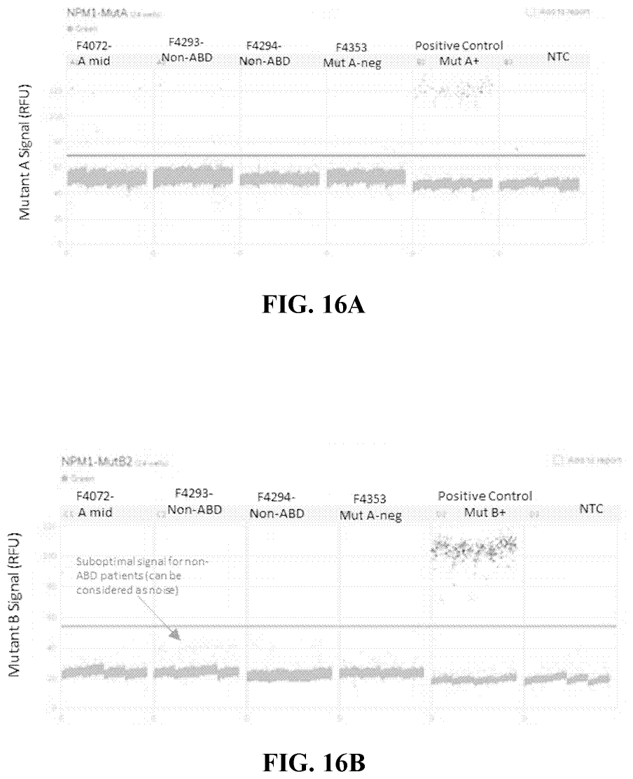

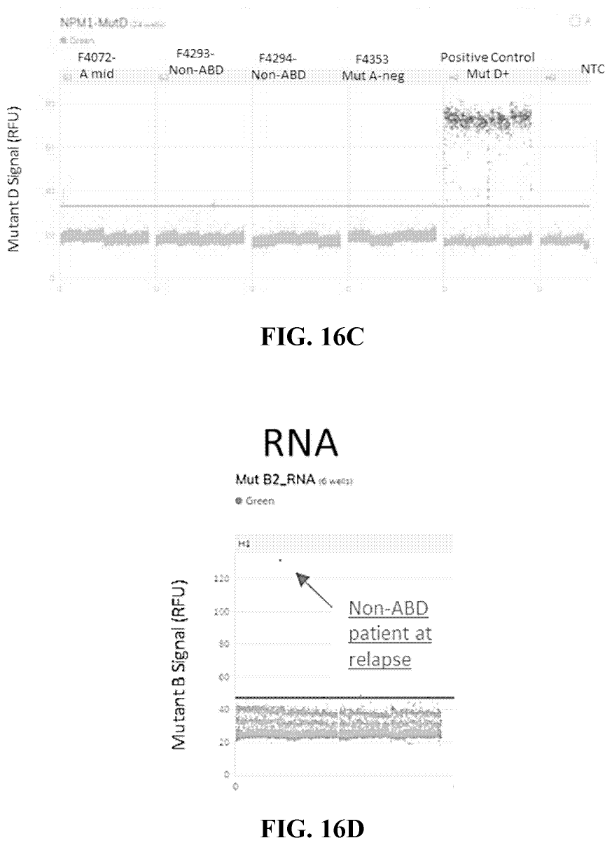

For non-ABD mutant, specificity to screen these patients was tested more extensively at DNA and briefly at RNA level, the major aim being to differentiate them from typical NPM1 mutations. As expected and shown in FIG. 16A-D, patient with non-ABD mutations showed a no or very poor signal intensity despite the use of dPCR analysis at both DNA and RNA level. This indicated that even a 1-mer mismatch significantly reduces the probe binding efficiency hence PCR is impeded.

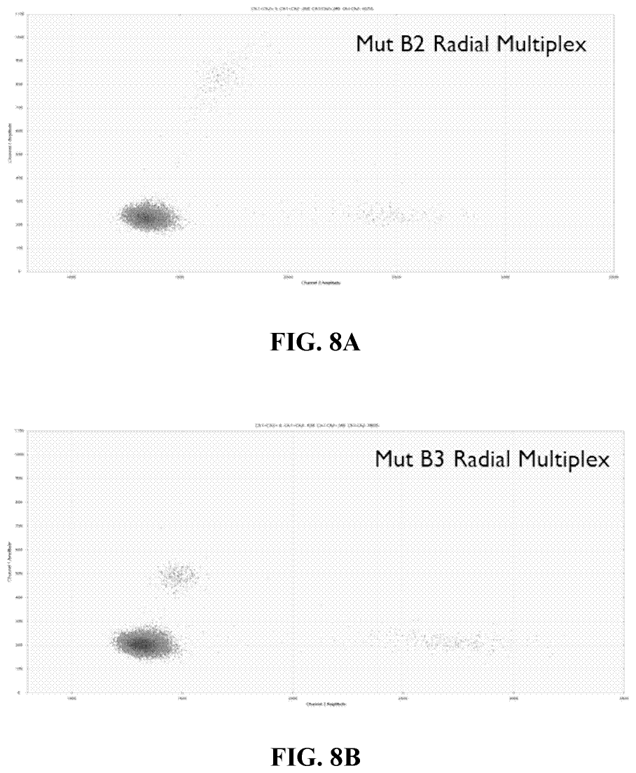

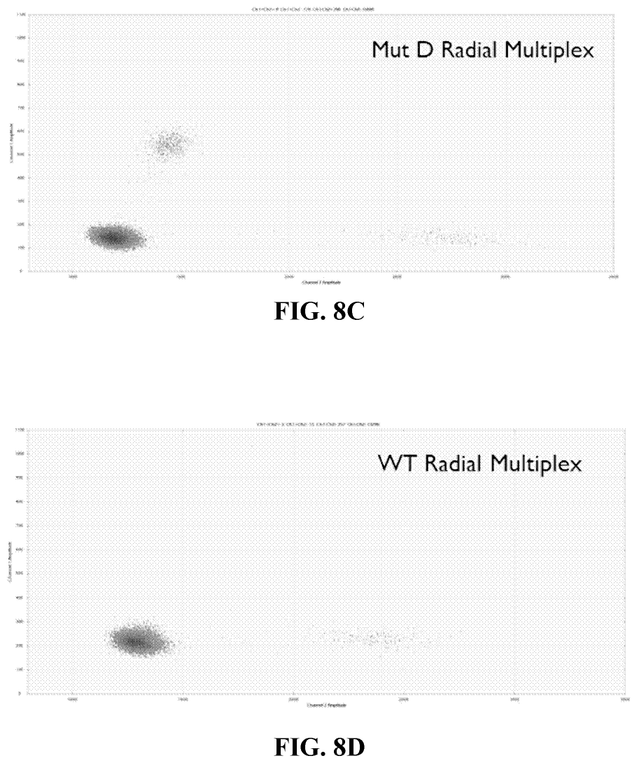

For genotype screening on QX200, the probes were further tested adopting a radial multiplexing approach in order to accommodate 4-plex detection on a 2-channel detection system in the final design. Each probe was labelled with both FAM and HEX and mixed at 1:1 ratio with an equal ratio of WT probe labelled with HEX. Using both channels (FAM and HEX) of QX200, Mut B and Mut D were further tested by both mixing FAM and HEX labelled probes at 1:1 ratio and mixed with WT labelled with HEX. As expected, in the 2D scatter plot shown in FIGS. 8A-8D, there was a diagonal shift of positive clusters of mutant signals. This result provided a solid base of the further optimization into a 4-plex of Mut A, B, D and WT in a single reaction well on QX200 or any platform with only two detection channels.

### Example 4: Quantification and Sensitivity Test

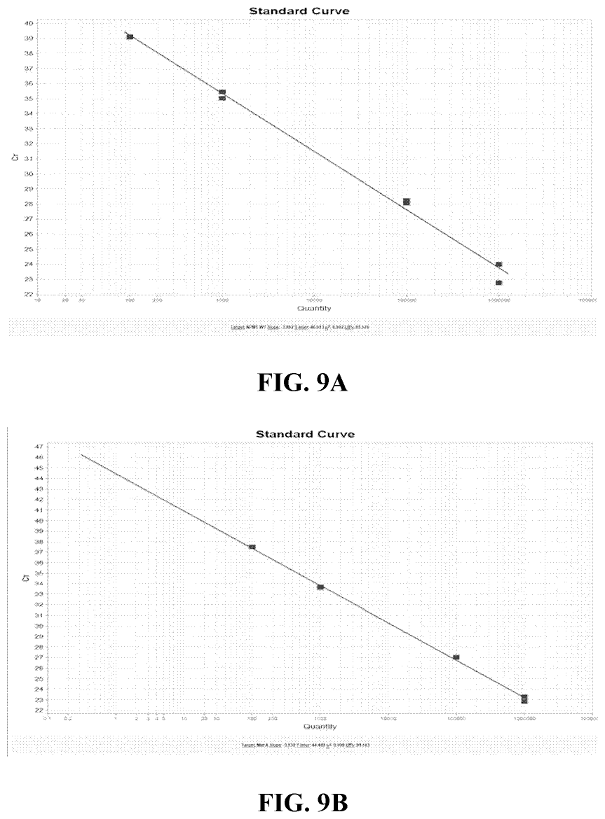

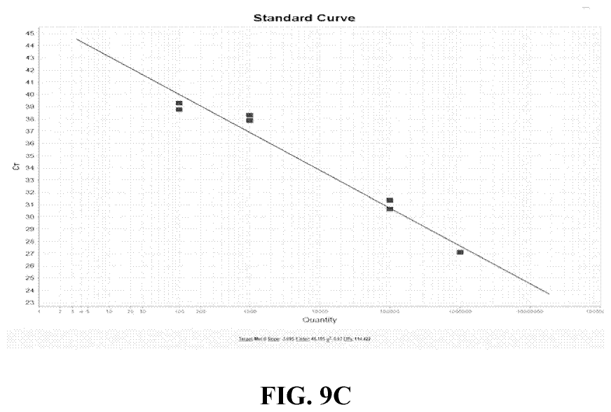

The absolute copy number of the mutants and WT NPM1 were determined either by the standard curve method (with serially diluted plasmid control) on Real Time PCR or by dPCR/ddPCR based on Poisson distribution without need of standard curve. These platforms currently shared the same PCR conditions for specificity, as mentioned above. The percentage of the mutant was then calculated as the ratio of the NPM1 mutant to the total NPM1 (mutant+WT) copy number and MRD was determined by comparing such ratio to baseline sample collected during diagnosis/earliest date available. FIGS. 9A-9C summarize the Real Time PCR optimization and shows the log linearity of calculated abundance (Ct values) with increasing dilution of Mut A, B and wild-type NPM1. Ideal linearity of the serial diluted plasmid was observed but the limit of detection was only 100 copies based on the current optimization. The sensitivity calculated of this assay was at least 100 copies. However, the sensitivity of Mut D could not be determined due to failed amplification in Real Time PCR and was pending repeat or with Mut D probe labelled with FAM.

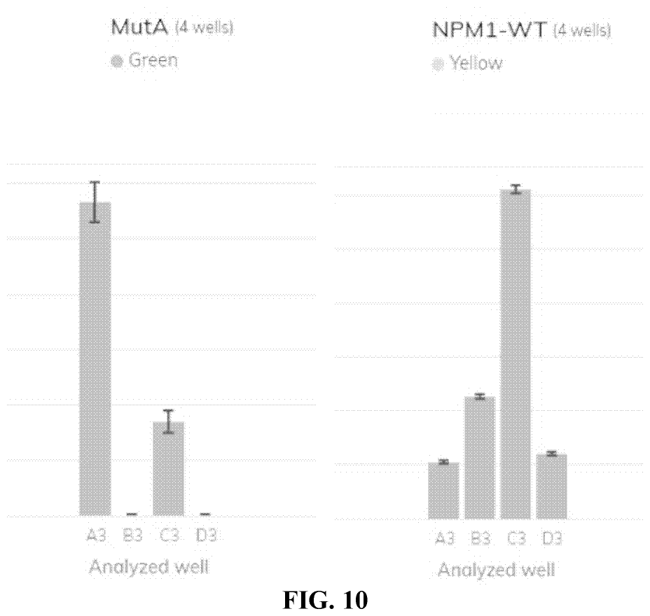

When testing the sensitivity on the dPCR Qiacuity system, the major focus for Mut A quantification was the correlation to in-house results done using RNA as input on the Real Time PCR platform. On the other hand, quantification of Mut B and D focused on the sensitivity when decreasing percentage of mutant plasmid was spiked into a constant amount of WT NPM1. As shown in FIG. 10 and in Table 2, complete concordance to the commercial RNA Real Time PCR kit (Ipsogen NPM1 Mut A PCR kit) was demonstrated in terms of MRD level. However, the mutation burden could not be compared in a similar manner due to technical differences between the quantification of RNA and DNA. Since the dPCR nanoplate is in theory capable of partitioning around 26,000 copies of DNA, measured copies of mutant plus WT copy number was lower than expected and further optimization of template loading was required. It was expected that increasing DNA input would increase the partition loading and hence further increase the sensitivity of quantification.

TABLE 2
Sensitivity test of dPCR for Mut A using human DNA samples and correlation
with in-house MRD determined using commercial RNA Real Time PCR kit

| | | | | | | | | Minimal |
|---|---|---|---|---|---|---|---|---|
| | Absolute | | | Concentration | | | Minimal | Residual |
| | count | | | (copies/μL) | | | Residual | Disease By |
| | | Wild | | | Wild | Mutation | Disease | RNA Real |
| | NPM1 | Type | Total | MutA | Type | Burden | by dPCR | Time PCR |
| Patient A | 236 | 1076 | 1312 | 11.3 | 52.5 | 17.988% | 1 | 1 |
| (F2628-pre treatment) | | | | | | | | |
| Patient A | 0 | 2190 | 2190 | 0 | 113.4 | 0.000% | 0.000% | 2.03 × 10⁻⁰⁵ |
| (F3435-post treatment) | | | | | | | | |
| Patient A | 67 | 5387 | 5454 | 3.4 | 305.6 | 1.228% | 1 | |
| (F2937-pre treatment) | | | | | | | | |
| Patient A | 0 | 1161 | 1161 | 0 | 60.4 | 0.000% | 0.000% | 2.46 × 10⁻³ |
| (F3458-post treatment) | | | | | | | | |

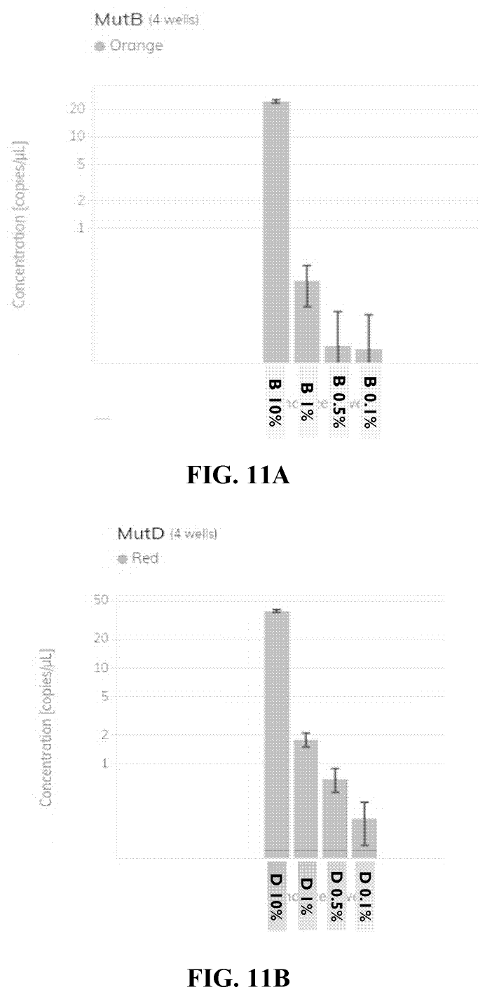

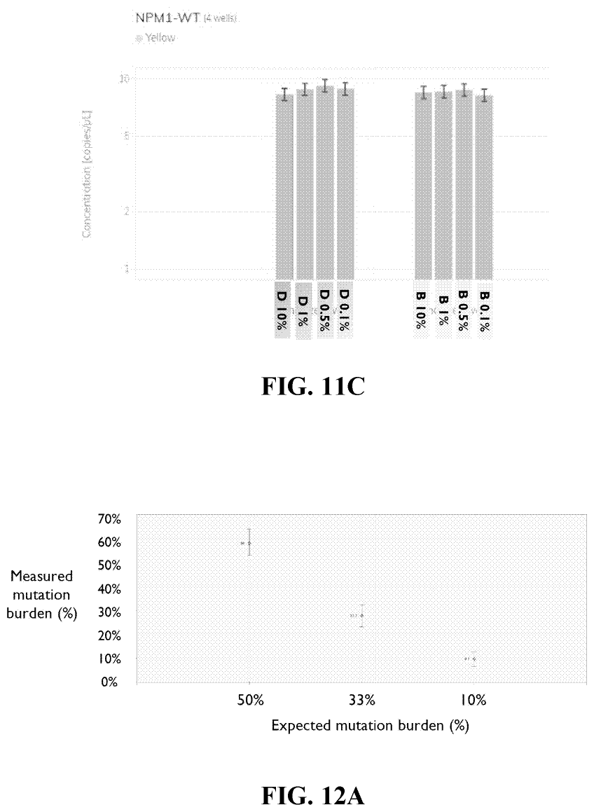

For the sensitivity test of Mut B labelled with TAMRA, and Mut D labelled with ROX which showed sub-optimal specificity during initial testing, quantification and sensitivity test was done by detecting mutant NPM1 under the presence of WT NPM1. This mimicked actual patient DNA which comprised of both leukemic cells and normal cells resulting in a DNA sample with both Mut and WT NPM1 gene (FIG. 11A-11C and Table 3).

TABLE 3
Sensitivity test of dPCR for Mut B and D using plasmid
control mimicking patient DNA of dPCR system (Qiacuity)

| | Valid Partition | Positive | Limit of Detection |
|---|---|---|---|
| B 10% | 25463 | 478 | |
| B 1% | 24756 | 5 | |
| B 0.5% | 25466 | 1 | |
| B 0.1% | 25436 | 1 | 3.93E−05 |
| D 10% | 25323 | 789 | |
| D 1% | 25443 | 35 | |
| D 0.5% | 24973 | 13 | |
| D 0.1% | 24785 | 5 | 2.02E−04 |

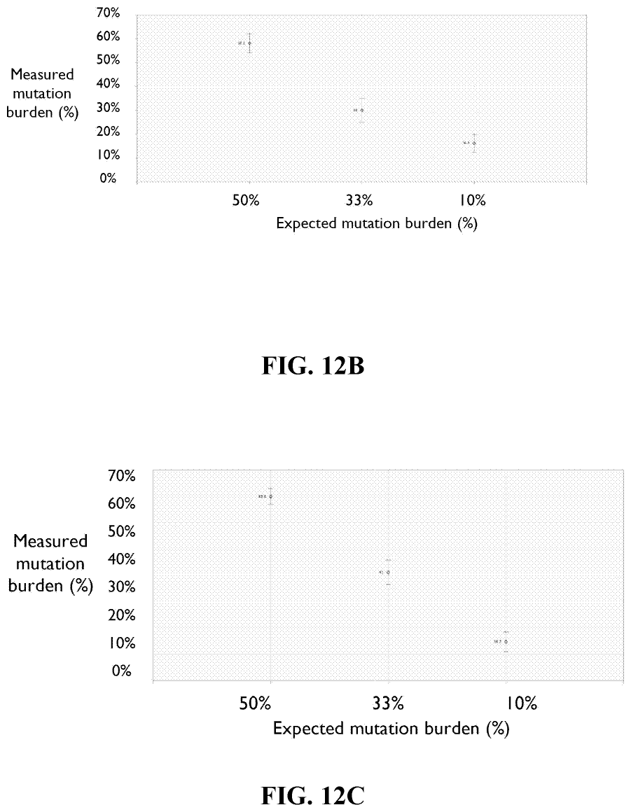

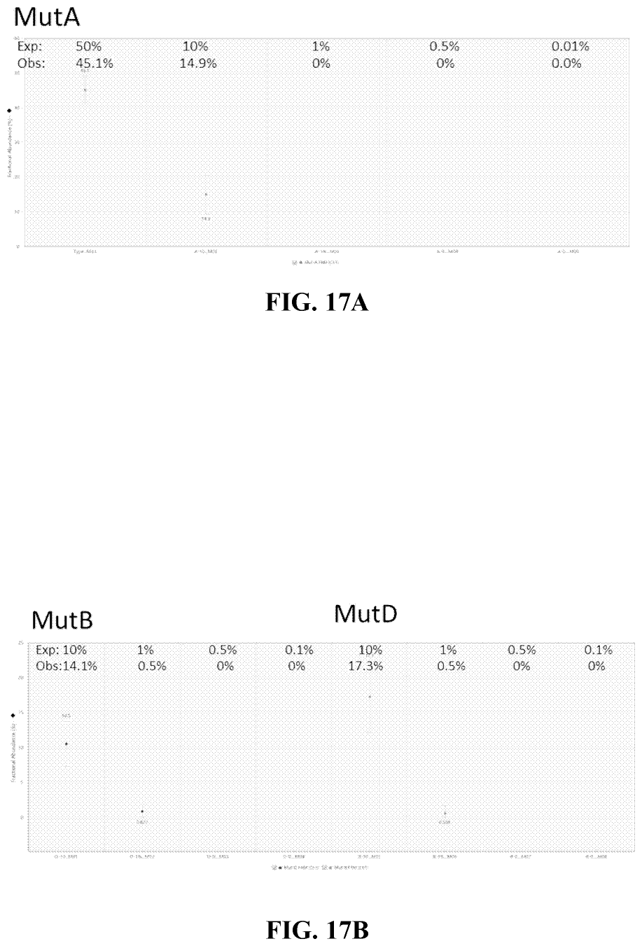

There was consistent positive detection of minute copies of both mutants and successfully counting even when only 0.1% (22 copies) of mutants were spiked into 22000 copies of WT NPM1. The log linearity of absolute count to actual spike-in copies was observed under a constant amount of WT copy was achieved. This indicated there was no or insignificant mutant probe inhibitions from WT probe even when WT DNA were abundant and could potentially dominate a PCR reaction. Similarly, when Mut B2, B3 and D probes was tested on QX200, a consistent detection of mutation burden was observed when different copies of Mut B or D were mixed with constant WT plasmid (20000 copies) ranging from 50%, 33% to 10% (FIGS. 12A-12C). All these results supported the proof-of-principle of using LNA probes to quantify NPM1 mutations for MRD purpose. With the new probe for Mut B, the LOD of all Mut probes was further tested on QX200 and as low as 1% mutant copy can be detected in 200 copy/uL of WT control at DNA level. With QX200 being the gold standard of digital PCR technology, the data shows that the disclosed test system has a LOD of Mut A being 10% and 1% for Mut B and D. (FIGS. 17A and 17B).

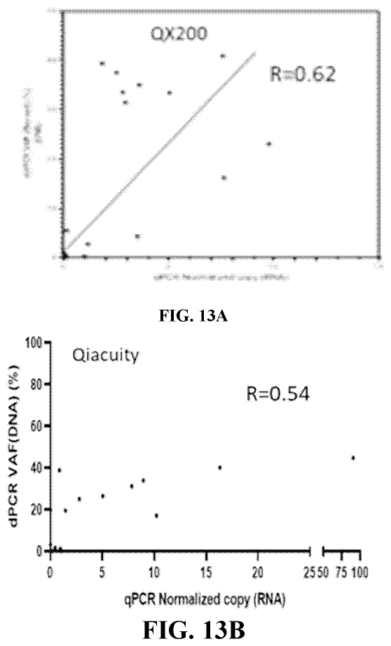

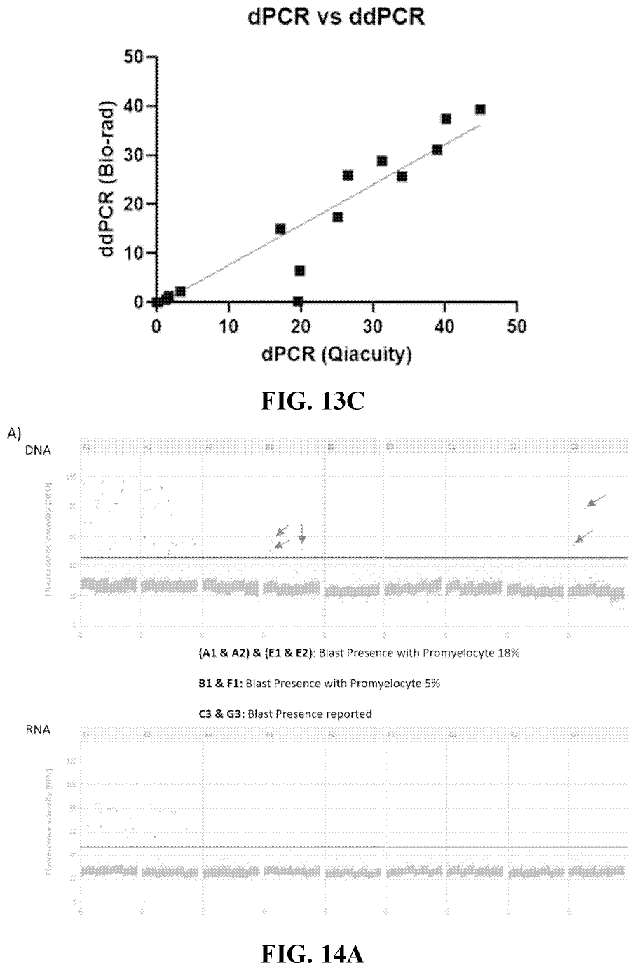

To test if the DNA platform can be an alternative to RNA assay which RNA has a more stringent requirements on patient specimen, paired DNA and RNA were studied by this in-house probe sets and compared with commercial Ipsogen RNA quantification assay FIG. 13 using both QX200 and Qiacuity and good correlation between two digital PCR platform but there is no significant correlation of DNA copy number compared with normalized copy number at RNA level which is normalized to ABL. Such difference is predicted attribute to the higher sensitivity of dPCR platform compared with Real Time PCR and the Poisson Distribution statistical method adopted by dPCR but not Real Time PCR.

Due to an insignificant correlation between DNA and RNA assay which dPCR is known to be more sensitive, other methods were adopted for downstream validation. This includes serial sample analysis of selected patients and grouped analysis using only digital PCR platforms.

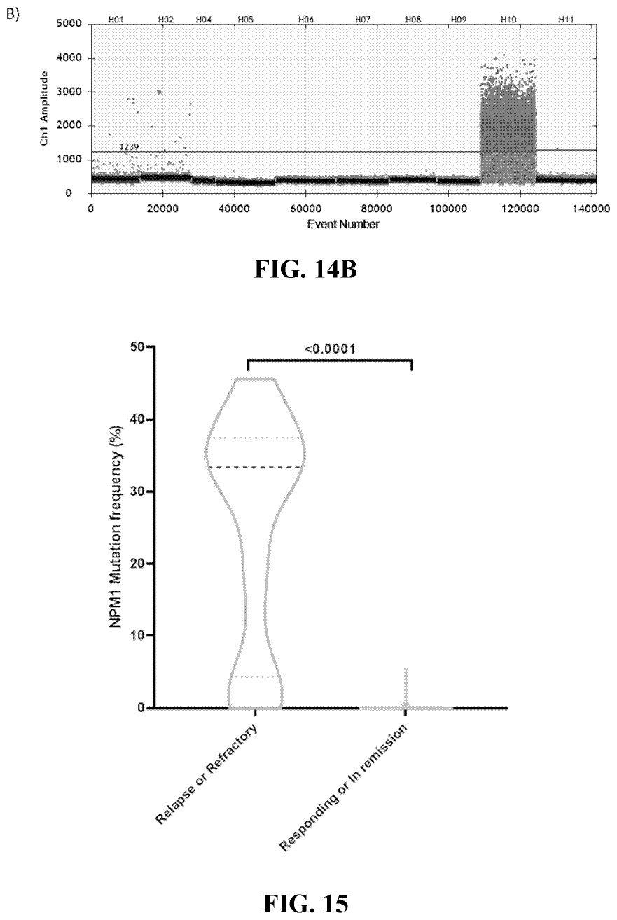

To test if the assay offers the expected sensitivity in detecting early molecular relapse in patients, several patients which have been observed with relapsed were studied at both DNA and RNA level. The Patient B which has the rare Mut B with 9 timepoints displaying applications of early molecular relapse monitoring in both DNA and RNA. Only 4 of the 9 timepoints have been documented with either a high percentage of promyelocyte count or the presence of blast detected. FIG. 14 shows in 4 of the 9 timepoints in the presence of blast cells or promyelocytes cells, copy numbers of NPM1 can be detected in both DNA and RNA with DNA displaying a higher sensitivity as compared to RNA. The performance of the assay is comparable between two different dPCR systems as shown in comparison between FIGS. 14A and 14B.

As shown in FIG. 15, patients DNA samples are grouped into two groups and studied if NPM1 mutation confer any clinical significance: (1) responding time points or during remission and (2) relapse and refractory to treatment including chemotherapy and HSCT. As predicted, patient time points at remission or under stable condition has a significantly lower NPM1 copy and the copy number is higher during disease relapse. These data collectively support the application of dPCR as a tool for MRD and both DNA and RNA could be used.

### CONCLUSIONS

To effectively screen for each of the three mutants with both specificity and sensitivity, a set probes sequences that can detect as low as 5 copies of mutants in dPCR and 100 copies in Real Time PCR were developed. Optimal specificity of each mutant probe provided the required specificity in both Real Time PCR and dPCR for screening at DNA level. Sensitivity on ddPCR is indicated to be comparable with dPCR. Sensitivity on Real Time PCR optimized using more DNA templates and modifying LNA bases position of the same probe sequences. Application on Real Time PCR can be achieved with monoplex PCR of each probe, due to WT probe dominance hence mutant probe limitation especially for low mutation burden sample.

For genotype screening at DNA level, dPCR clearly showed that the 4-plex PCR system could serve as a robust screening tool for NPM1 mutations. The suboptimal specificity for probe detecting Mut B in Real Time PCR and sensitivity to screen for Mut D are solved by new probe Mut B2 and B3 and Mutant D labelled with FAM. For enhanced sensitivity of genotyping on Real Time PCR, monoplex PCR can be used; however, RNA assay has been introduced. In terms of dye modifications, the stronger alternative dye combinations for Mut B, D and wild type NPM1 are ATTO550, TYE665 and Yakima Yellow respectively. FAM for Mut A is already a strong dye hence Mut A probe optimization will focus on LNA modifications. Other stronger dyes are also available.

To be compatible with dPCR or ddPCR equipment with limited fluorescent detectors (e.g., green and yellow detectors), the 4-colour system is also modified to provide another version by using only two fluorescent dyes FAM and HEX/Yakima Yellow for green and yellow detectors following the radial multiplexing approach. For this method, two probes each labelled with FAM and HEX/Yakima Yellow are used for mutants B and D. During PCR, these four probes are mixed in different ratios (1:3 and 3:1 ratio respectively) alongside the FAM probes for Mut A and HEX probes for WT NPM1. As a result, a mixed signal of Mut B and D is detectable by sharing the same detectors of Mut A and WT NPM1 without overlapping position on the 2-D scatter plot in the dPCR or ddPCR systems.

The systems for Screening for mutations in NPM1 gene also been designed with derivative version with the forward PCR primer binding the end of exon 11 while reverse primer span exon 12 downstream to the mutations. This enables the detection of mutations using cDNA converted from RNA as input for screening purpose. Similar to DNA assay, a consistent specificity and sensitivity hence application as MRD tools is highly recommended as replacement of conventional Real Time PCR and dPCR assays using unmodified probes.

The invention will be further understood by virtue of the following numbered paragraphs.

1. A method for detecting a presence, absence, and/or level of mutation in Exon 12 of the NPM1 gene in a sample, comprising:
(a) hybridizing labelled sequence probes to nucleic acid of Exon 12 of the NPM1 gene in a sample, to form probe-nucleic acid conjugates, wherein the hybridizing comprises:
   (i) combining in a reaction mixture under primer extension conditions a set of two oligonucleotide primers with the sample,
   wherein the primers each comprise a nucleic acid sequence complementary to a nucleic acid sequence of the NPM1 gene and are each configured to produce an extension product comprising all or part of Exon 12 of the NPM1 gene,
   (ii) adding to the reaction mixture one or more labelled sequence probes,
   wherein each probe comprises (1) a nucleic acid sequence complementary to a nucleic acid sequence of the wild-type NPM1 gene or of a mutant of the NPM1 gene, (2) one or more Locked Nucleic Acid (LNA) residues, and (3) a detectable label,
   wherein the nucleic acid sequence of the probe complementary to the NPM1 gene anneals to the extension product at a predetermined location that is only present in the wild-type NPM1 gene or in the mutant of the NPM1 gene; and
(b) detecting the detectable label,
wherein detection of the detectable labels informs the presence, absence and/or level of NPM1 mutations in the sample.

2 The method of paragraph 1, wherein the mutation of the NPM1 gene is one or more frameshift mutations selected from the group consisting of type A, type B, and type D mutations of the NPM1 gene.

3. The method of paragraph 1 or paragraph 2, wherein the extension product comprises all or part of one of SEQ ID NOs: 1-4.

4. The method of any one of paragraphs 1-3, wherein the set of two oligonucleotide primers comprises the nucleic acid sequences of SEQ ID NO:5 and SEQ ID NO:6.

5. The method of any one of paragraphs 1-3, wherein the set of two oligonucleotide primers comprises the nucleic acid sequences of SEQ ID NO:7 and SEQ ID NO:8.

6. The method of any one of paragraph s 1-5, wherein the primer extension step comprises performing a polymerase chain reaction (PCR) in the reaction mixture to form an amplicon using Real Time PCR, or Digital PCR, or Droplet-Digital PCR.

7. The method of any one of paragraphs 1-6, wherein the sample comprises a nucleic acid vector comprising all or part of the NPM1 gene, genomic DNA, cDNA, or combinations thereof.

8. The method of any one of claims 1-7, further comprising
(d) recording the presence, absence and/or level of NPM1 mutations in the sample.

9 The method of paragraph 8, wherein the method further comprises the use of one or more control samples.

10. The method of paragraph 9, wherein recording the presence, absence, and/or level of NPM1 mutations in the sample comprises comparing the results from the sample with those obtained from the one or more controls.

11. The method of paragraph 9, wherein the results are compared with four controls, comprising a first control nucleic acid sequence corresponding to wild-type NPM1, a second control nucleic acid sequence corresponding to type A NPM1 mutation, a third control nucleic acid sequence corresponding to type B NPM1 mutation, and a fourth control nucleic acid sequence type D NPM1 mutation.

12. The method of paragraph 9 or 10, wherein the controls comprise a nucleic acid sequence of one or more of SEQ ID NOs: 1-4 and SEQ ID NOs: 9-12.

13. The method of any one of paragraphs 1-12, wherein the detectable label comprises a fluorescent dye and a quencher.

14. The method of paragraph 13, wherein the fluorescent dye is attached to the 5′ end of the probe and the dark quencher is attached to the 3′ end of the probe.

15. The method of paragraph 13 or paragraph 14, wherein the fluorescent dye is selected from the group consisting of 6-FAM, Fluorescein dT, Cy3™, 5 (6)-Carboxytetramethylrhodamine, JOE, Cy5™, MAX, TET™, TEX615, TYE665, 6-ROX, Cy5.5™, YAKIMA YELLOW™, TEX615, TYE665, TYE705, SUN, ATTO488, ATTO532, ATTO550, ATTO565, ATTORHO101, ATTO590, ATTO633, ATTO647N, HEX, and Alexa Fluor dyes.

16. The method of any one of paragraphs 1-15, wherein the labelled sequence probes comprise one, two, three, or four probes, wherein the probes are selected from the group consisting of a first probe that selectively hybridizes to nucleic acid of wild-type NPM1, a second probe that selectively hybridizes to nucleic acid of type A NPM1 mutation, a third probe that selectively hybridizes to nucleic acid of type B NPM1 mutation, and a fourth probe that selectively hybridizes to nucleic acid of type D NPM1 mutation.

17. The method of paragraph 16, wherein the nucleic acid sequence of the first probe comprises SEQ ID NO:17 or SEQ ID NO:22, wherein the nucleic acid sequence of the second probe comprises SEQ ID NO:18 or SEQ ID NO:23, wherein the nucleic acid sequence of the third probe comprises SEQ ID NO: 19 or SEQ ID NO:20, and wherein the nucleic acid sequence of the fourth probe comprises SEQ ID NO:21.

18. The method of any one of paragraphs 1-17, wherein the method requires at least 100 copies, or less than 100 copies of NPM1 DNA.

19. The method of paragraph 18, wherein the method requires at least 10 copies, or less than 10 copies of NPM1 DNA.

20. The method of paragraph 19, wherein the method requires at least 5 copies, or less than 5 copies of NPM1 DNA.

21. The method of any one of paragraphs 1-20, wherein the sample is from an individual suffering from leukemia, or who is identified as being at risk of a leukemia.

22. The method of paragraph 21, wherein the subject has Acute Myeloid Leukemia (AML), or is identified as being at risk of AML.

23. The method of paragraph 21 or paragraph 22, further comprising the step of administering to the subject a therapeutic agent or procedure.

24. The method of any one of paragraphs 21 to 23, wherein the subject is undergoing, or is eligible for hematopoietic stem cell therapy (HSCT).

25. The method of any one of paragraphs 1-24, wherein the method is used to detect or quantify Measurable Residual Disease (MRD) in the subject.

26. A labelled sequence probe for use in a method any one of paragraphs 1-25.

27. A kit for detecting a presence, absence, and/or level of mutation of the NPM1 gene in a sample, comprising
(i) oligonucleotide primers,
wherein the primers each comprise a nucleic acid sequence complementary to a nucleic acid sequence of the NPM1 gene and are each configured to produce an extension product comprising all or part of the NPM1 gene; and
(ii) one or more labelled sequence probes,
wherein the probes each comprise Locked nucleic Acid (LNA) having a sequence complementary to a nucleic acid sequence of the NPM1 gene or of a mutant of the NPM1 gene, a reporter dye, and a dark quencher,
wherein the nucleic acid sequence complementary to the NPM1 gene anneals to the extension product at a predetermined location; and
(iii) instructions for the method of claim 1.

1. A method for detecting a presence, absence, and/or level of mutation in Exon 12 of the NPM1 gene in a sample, comprising:
(a) hybridizing labelled sequence probes to nucleic acid of Exon 12 of the NPM1 gene in a sample, to form probe-nucleic acid conjugates,
wherein the hybridizing comprises:
(i) combining in a reaction mixture under primer extension conditions a set of two oligonucleotide primers with the sample,
wherein the primers each comprise a nucleic acid sequence complementary to a nucleic acid sequence of the NPM1 gene and are each configured to produce an extension product comprising all or part of Exon 12 of the NPM1 gene,
(ii) adding to the reaction mixture one or more labelled sequence probes,
wherein each probe comprises (1) a nucleic acid sequence complementary to a nucleic acid sequence of the wild-type NPM1 gene or of a mutant of the NPM1 gene, (2) one or more Locked nucleic Acid (LNA) residues, and (3) a detectable label,
wherein the nucleic acid sequence of the probe complementary to the NPM1 gene anneals to the extension product at a predetermined location that is only present in the wild-type NPM1 gene or in the mutant of the NPM1 gene; and
(b) detecting the detectable label,
wherein detection of the detectable labels informs the presence, absence and/or level of NPM1 mutations in the sample.

2. The method of claim 1, wherein the mutation of the NPM1 gene is one or more frameshift mutations selected from the group consisting of type A, type B, and type D mutations of the NPM1 gene.

3. The method of claim 1, wherein the extension product comprises all or part of one of SEQ ID NOs: 1-4.

4. The method of claim 1, wherein the set of two oligonucleotide primers comprises the nucleic acid sequences of (a) SEQ ID NO:5 and SEQ ID NO: 6; or (b) SEQ ID NO:7 and SEQ ID NO:8).

5. (canceled)

6. The method of claim 1, wherein amplifying the probe-nucleic acid conjugates in (b) comprises performing a polymerase chain reaction (PCR) in the reaction mixture to form an amplicon using Real Time PCR, or Digital PCR, or Droplet-Digital PCR.

7. The method of claim 1, wherein the sample comprises a nucleic acid vector comprising all or part of the NPM1 gene, genomic DNA, cDNA, or combinations thereof and/or the method further comprising
(d) recording the presence, absence and/or level of NPM1 mutations in the sample.

8. (canceled)

9. The method of claim 8, wherein the method further comprises the use of one or more control samples.

10. The method of claim 9, wherein: (a) recording the presence, absence, and/or level of NPM1 mutations in the sample comprises comparing the results from the sample with those obtained from the one or more controls; or (b) the results are compared with four controls, comprising a first control nucleic acid sequence corresponding to wild-type NPM1, a second control nucleic acid sequence corresponding to type A NPM1 mutation, a third control nucleic acid sequence corresponding to type B NPM1 mutation, and a fourth control nucleic acid sequence type D NPM1 mutation.

11. (canceled)

12. The method of claim 9, wherein the controls comprise a nucleic acid sequence of one or more of SEQ ID NOs: 1-4 and SEQ ID NOs: 9-12.

13. The method of claim 1, wherein the detectable label comprises a fluorescent dye and a quencher and/or the fluorescent dye is attached to the 5′ end of the probe and the dark quencher is attached to the 3′ end of the probe.

14. (canceled)

15. The method of claim 13, wherein the fluorescent dye is selected from the group consisting of 6-FAM, Fluorescein dT, Cy3™, 5(6)-Carboxytetramethylrhodamine, JOE, Cy5™, MAX, TET™, TEX615, TYE665, 6-ROX, Cy5.5™, YAKIMA YELLOW™, TEX615, TYE665, TYE705, SUN, ATTO488, ATTO532, ATTO550, ATTO565, ATTORHO101, ATTO590, ATTO633, ATTO647N, HEX, and Alexa Fluor dyes.

16. The method of claim 1, wherein the labelled sequence probes comprise one, two, three, or four probes, wherein the probes are selected from the group consisting of a first probe that selectively hybridizes to nucleic acid of wild-type NPM1, a second probe that selectively hybridizes to nucleic acid of type A NPM1 mutation, a third probe that selectively hybridizes to nucleic acid of type B NPM1 mutation, and a fourth probe that selectively hybridizes to nucleic acid of type D NPM1 mutation.

17. The method of claim 16, wherein the nucleic acid sequence of the first probe comprises SEQ ID NO:17 or SEQ ID NO:22, wherein the nucleic acid sequence of the second probe comprises SEQ ID NO:18 or SEQ ID NO:23, wherein the nucleic acid sequence of the third probe comprises SEQ ID NO:19 or SEQ ID NO:20, and wherein the nucleic acid sequence of the fourth probe comprises SEQ ID NO:21.

18. The method of claim 1, wherein the method requires at least 100 copies, or less than 100 copies of NPM1 DNA.

19. The method of claim 18, wherein the method requires at least 10 copies, or less than 10 copies of NPM1 DNA.

20. The method of claim 19, wherein the method requires at least 5 copies, or less than 5 copies of NPM1 DNA.

21. The method of claim 1, wherein the sample is from an individual suffering from leukemia, or who is identified as being at risk of a leukemia.

22. The method of claim 21, (a) wherein the subject has Acute Myeloid Leukemia (AML), or is identified as being at risk of AML; (b), further comprising the step of administering to the subject a therapeutic agent or procedure; and/or (c) wherein the subject is undergoing, or is eligible for hematopoietic stem cell therapy (HSCT).

23. (canceled)

24. (canceled)

25. The method of claim 1, wherein the method is used to detect or quantify Measurable Residual Disease (MRD) in the subject.

26. A labelled sequence probe for use in a method claim 1, optionally, in a kit for detecting a presence, absence, and/or level of mutation of the NPM1 gene in a sample, comprising
(i) oligonucleotide primers,
wherein the primers each comprise a nucleic acid sequence complementary to a nucleic acid sequence of the NPM1 gene and are each configured to produce an extension product comprising all or part of the NPM1 gene; and
(ii) one or more labelled sequence probes,
wherein the probes each comprise Locked nucleic Acid (LNA) having a sequence complementary to a nucleic acid sequence of the NPM1 gene or of a mutant of the NPM1 gene, a reporter dye, and a dark quencher,
wherein the nucleic acid sequence complementary to the NPM1 gene anneals to the extension product at a predetermined location; and
(iii) instructions for the method of claim 1.

27. (canceled)
